(19) United States
(12) Patent Application Publication
(10) Pub. No.: US 2022/0125773 A1
(43) Pub. Date: Apr. 28, 2022

(54) AQUEOUS FORMULATIONS OF WATER INSOLUBLE COX-2 INHIBITORS

(71) Applicant: Tremeau Pharmaceuticals, Inc., Concord, MA (US)

(72) Inventors: Bradford C. SIPPY, Acton, MA (US); Raymond D. SKWIERCZYNSKI, Andover, MA (US)

(21) Appl. No.: 17/512,268

(22) Filed: Oct. 27, 2021

Related U.S. Application Data
(60) Provisional application No. 63/106,571, filed on Oct. 28, 2020.

(51) Int. Cl. A61K 31/444; A61K 9/08; A61K 31/415; A61K 31/365; A61K 47/40; A61K 9/19; A61K 9/00

(52) U.S. Cl. CPC A61K 31/444; A61K 9/08; A61K 31/415; A61K 9/0053; A61K 47/40; A61K 9/19; A61K 31/365

(57) ABSTRACT

Described herein are compositions and methods for treatment with aqueous formulations of water insoluble COX-2 inhibitors and a solubilizing agent.

This application claims the benefit of and priority to U.S. Provisional Patent Application No. 63/106,571, filed on Oct. 28, 2020, the content of which is hereby incorporated by reference in its entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Analgesics to control postoperative pain can be administered immediately prior to surgery (preoperatively), during surgery (intraoperatively), after surgery (postoperatively), or following hospital discharge via a prescription. Commonly used drug classes include opioids, acetaminophen, nonselective NSAIDs, and local anesthetics. Currently, the market for intravenously administered analgesics is dominated by two agents: IV ketorolac (TORADOL) and IV acetaminophen (OFIRMEV). IV ketorolac is widely used because of its strong efficacy profile, but it carries several important limitations, including a significant risk of serious gastrointestinal (GI) bleeds, an indication that is limited to short-term (<5 days) use in adults only, and a contra-indication for pre-operative use, due to its effect on platelet aggregation. IV acetaminophen, on the other hand, is recognized as safer than IV ketorolac, but is limited in use due to its modest efficacy at treating pain. NSAIDs have pain-relieving, antipyretic and anti-inflammatory properties, are proven to be effective following day surgery and minor surgery, and have an opiate-sparing effect after more major surgery. However, a major concern regarding the use of conventional NSAIDs postoperatively is the possibility of bleeding from both the operative site (because of the inhibition of platelet aggregation) and from the upper GI tract (especially in patients stressed by surgery, the elderly, frail, or dehydrated). There is a significant but unmet need for intravenously administered drugs that combine the pain-relieving properties of NSAIDs without these adverse effects, especially to control postoperative pain.

COX-2 inhibitors are a subclass of nonsteroidal anti-inflammatory drugs (NSAIDs) that have been shown to be highly effective in treating conditions such as acute pain, and may be administered to patients with a high risk of operative site bleeding, upper GI bleeding or those with a history of peptic ulcer. Few, if any, COX-2 selective NSAIDs are available in a formulation that is suitable for use in an intravenous or other parenteral administration, due to the fact that they are insoluble or practically insoluble in water. These water insoluble COX-2 inhibitors include rofecoxib, etoricoxib, and celecoxib. Described herein are aqueous formulations of water insoluble COX-2 inhibitors suitable for oral or intravenous or other parenteral administration.

### SUMMARY OF THE INVENTION

In certain aspects, the subject matter disclosed herein provides a pharmaceutical composition comprising an aqueous solution comprising: a) water, b) a water insoluble COX-2 inhibitor or a pharmaceutically acceptable salt, ester or co-crystal thereof, and c) a solubilizing agent, wherein the solubility of the water insoluble COX-2 inhibitor in the solution is more than 10 μg/ml.

In some embodiments, the water insoluble COX-2 inhibitor is selected from the group consisting of rofecoxib, etoricoxib, and celecoxib. In some embodiments, the water insoluble COX-2 inhibitor is rofecoxib. In some embodiments, the composition further comprises at least one co-solvent helper. In some embodiments, the composition further comprises at least one antioxidant. In some embodiments, the composition further comprises at least one buffering agent. In some embodiments, the composition further comprises at least one isotonic agent.

In some embodiments, said composition is a reconstituted lyophile. In some embodiments, said pharmaceutical composition is diluted prior to administration.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the β-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the effective amount of the water insoluble COX-2 inhibitor in a single dose formulation is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the concentration % (w/v) of the water insoluble COX-2 inhibitor in the solution is selected from the group consisting of about 0.001%, about 0.002%, about 0.003%, about 0.004%, about 0.005%, about 0.006%, about 0.007%, about 0.008%, about 0.01%, about 0.012%, about 0.015%, about 0.017%, about 0.02%, about 0.025%, about 0.03%, about 0.035%, about 0.04%, about 0.05%, about 0.06%, about 0.07% and any other suitable concentration. In some embodiments, the volume of the solution is selected from the group consisting of about 5 mL, about 10 ml, about 20 ml, about 25 mL, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 180 ml, and any suitable volume. In some embodiments, the solubility of the water insoluble COX-2 inhibitor in the solution is more than 20 μg/ml, more than 30 μg/ml, more than 40 μg/ml, more than 50 μg/ml, more than 60 μg/ml, more than 70 μg/ml, more than 80 μg/ml, more than 90 μg/ml, more than 100 μg/ml, more than 110 μg/ml, more than 120 μg/ml, more than 130 μg/ml, more than 140 μg/ml, or more than 150 μg/ml.

In some embodiments, said composition is administered as part of a combination therapy with at least one other therapeutic agent. In some embodiments, said composition is suitable for intravenous administration to a subject.

In certain aspects, the subject matter disclosed herein provides a lyophilized pharmaceutical composition comprising a lyophilization product of any of the pharmaceutical compositions described herein.

In certain aspects, the subject matter disclosed herein provides a method for treatment of pain, fever, or inflammation in a subject in need thereof, the method comprising parenterally administering to the subject a therapeutically effective amount of a pharmaceutical composition comprising an aqueous solution comprising: a) water, b) a COX-2 inhibitor or a pharmaceutically acceptable salt or ester thereof, and c) a solubilizing agent.

In some embodiments, the water insoluble COX-2 inhibitor is selected from the group consisting of rofecoxib, etoricoxib, and celecoxib. In some embodiments, the water insoluble COX-2 inhibitor is rofecoxib.

In some embodiments, the composition further comprises at least one co-solvent helper. In some embodiments, the composition further comprises at least one antioxidant. In some embodiments, the composition further comprises at least one buffering agent. In some embodiments, the composition further comprises at least one isotonic agent. In some embodiments, said composition is a reconstituted lyophile. In some embodiments, said pharmaceutical composition is diluted.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the β-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the effective amount of the COX-2 inhibitor in a single dose formulation is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the concentration % (w/v) of the COX-2 inhibitor in an aqueous formulation is selected from the group consisting of about 0.001%, about 0.002%, about 0.003%, about 0.004%, about 0.005%, about 0.006%, about 0.007%, about 0.008%, about 0.01%, about 0.012%, about 0.015%, about 0.017%, about 0.02%, about 0.025%, about 0.03%, about 0.035%, about 0.04%, about 0.05%, about 0.06%, about 0.07% and any other suitable concentration. In some embodiments, the volume of an aqueous formulations of the COX-2 inhibitor is selected from the group consisting of about 1 ml, about 2 ml, about 3 ml, about 4 ml, about 5 ml, about 10 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 180 ml, and any suitable volume. In some embodiments, the solubility of the COX-2 inhibitor is enhanced to more than 10 μg/ml, more than 20 μg/ml, more than 30 μg/ml, more than 40 μg/ml, more than 50 μg/ml, more than 60 μg/ml, more than 70 μg/ml, more than 80 μg/ml, more than 90 μg/ml, more than 100 μg/ml, more than 110 μg/ml, more than 120 μg/ml, more than 130 μg/ml, more than 140 μg/ml, or more than 150 μg/ml. In some embodiments, said composition is administered as part of a combination therapy with at least one other therapeutic agent.

In certain aspects, the subject matter disclosed herein provides a lyophilized pharmaceutical composition comprising a water insoluble COX-2 inhibitor or a pharmaceutically acceptable salt, ester or co-crystal thereof and a solubilizing agent, wherein the lyophilized pharmaceutical composition is a produced by lyophilizing a pharmaceutical composition comprising an aqueous formulation comprising water, a water insoluble COX-2 inhibitor or a pharmaceutically acceptable salt, ester or co-crystal thereof, and a solubilizing agent.

In certain aspects, the subject matter disclosed herein provides a pharmaceutical composition comprising an oral solution comprising: a) a COX-2 inhibitor or a pharmaceutically acceptable salt, ester or co-crystal thereof, and b) a solubilizing agent. In some embodiments, the COX-2 inhibitor is a water insoluble COX-2 inhibitor.

In some embodiments, the COX-2 inhibitor is selected from the group consisting of rofecoxib, etoricoxib, and celecoxib. In some embodiments, the COX-2 inhibitor is rofecoxib. In some embodiments, the composition further comprises at least one co-solvent helper. In some embodiments, the composition further comprises at least one antioxidant. In some embodiments, the composition further comprises at least one buffering agent. In some embodiments, the pharmaceutical composition is diluted prior to administration.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the 3-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the pharmaceutical composition comprises an effective amount of the COX-2 inhibitor in a single dose, wherein the effective amount is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the pharmaceutical composition has a volume of the oral solution selected from the group consisting of about 5 ml, about 10 ml, about 15 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 165 ml, about 170 ml, about 175 ml, about 180 ml, about 185 ml, about 190 ml, about 200 ml, or any suitable volume. In some embodiments, the pharmaceutical composition the oral solution comprises of about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about 0.14 mg/ml, or about 0.15 mg/ml of rofecoxib.

In some embodiments, the pharmaceutical composition is administered as part of a combination therapy with at least one other therapeutic agent. In some embodiments, the pharmaceutical composition is suitable for oral administration to a subject.

In some embodiments, the oral solution comprises rofecoxib and achieves a geometric mean plasma AUC_{0-48 hr} from about 3053 to about 4772 h*ng/ml following administration of a single dose of the oral solution to a population of healthy adults less than 65 years of age. In some embodiments, the geometric mean plasma AUC_{0-48 hr} is about 3053 h*ng/ml, about 3100 h*ng/ml, about 3200 h*ng/ml, about 3300 h*ng/ml, about 3400 h*ng/ml, about 3500 h*ng/ml, about 3600 h*ng/ml, about 3700 h*ng/ml, about 3800 h*ng/ml, about 3900 h*ng/ml, about 4000 h*ng/ml, about 4100 h*ng/ml, about 4200 h*ng/ml, about 4300 h*ng/ml, about 4320 h*ng/ml, about 4500 h*ng/ml, about 4600 h*ng/ml, about 4700 h*ng/ml, or about 4772 h*ng/ml. In some embodiments, the oral solution comprising rofecoxib achieves a plasma AUC_{0-48 hr} of between 174.5 h*ng/ml and 276 h*ng/ml for each 1 mg of rofecoxib in the solution.

In some embodiments, the oral solution comprises rofecoxib and achieves a geometric mean plasma Cₘₐₓ from about 276 to about 432 ng/ml following administration of a single dose of the oral solution to a population of healthy adults less than 65 years of age. In some embodiments, the geometric mean plasma Cₘₐₓ is about 276 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, or about 432 ng/ml. In some embodiments, the oral solution comprising rofecoxib achieves a Cₘₐₓ of between 16 ng/ml and 25.1 ng/ml for each 1 mg of rofecoxib in the solution.

In certain aspects, the subject matter disclosed herein provides a method for treatment of pain, fever, or inflammation in a subject in need thereof, the method comprising administering to the subject a therapeutically effective amount of a pharmaceutical composition comprising an oral solution comprising: a) a COX-2 inhibitor or a pharmaceutically acceptable salt, ester or co-crystal thereof, and b) a solubilizing agent. In some embodiments, the COX-2 inhibitor is a water insoluble COX-2 inhibitor.

In some embodiments, the COX-2 inhibitor is selected from the group consisting of rofecoxib, etoricoxib, and celecoxib. In some embodiments, the COX-2 inhibitor is rofecoxib. In some embodiments, the composition further comprises at least one co-solvent helper. In some embodiments, the composition further comprises at least one antioxidant. In some embodiments, the composition further comprises at least one buffering agent. In some embodiments, the pharmaceutical composition is diluted prior to administration.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the 3-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the method comprises an effective amount of the COX-2 inhibitor in a single dose selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the oral solution has a volume selected from the group consisting of about 5 ml, about 10 ml, about 15 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 165 ml, about 170 ml, about 175 ml, about 180 ml, about 185 ml, about 190 ml, about 200 ml, or any suitable volume. In some embodiments, the oral solution comprises about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about 0.14 mg/ml, or about 0.15 mg/ml of rofecoxib. In some embodiments, the composition is administered as part of a combination therapy with at least one other therapeutic agent.

In some embodiments, the oral solution comprises rofecoxib and achieves a geometric mean plasma AUC_{0-48 hr} from about 3053 to about 4772 h*ng/ml following administration of a single dose of the oral solution to a population of healthy adults less than 65 years of age. In some embodiments, the geometric mean plasma AUC_{0-48 hr} is about 3053 h*ng/ml, about 3100 h*ng/ml, about 3200 h*ng/ml, about 3300 h*ng/ml, about 3400 h*ng/ml, about 3500 h*ng/ml, about 3600 h*ng/ml, about 3700 h*ng/ml, about 3800 h*ng/ml, about 3900 h*ng/ml, about 4000 h*ng/ml, about 4100 h*ng/ml, about 4200 h*ng/ml, about 4300 h*ng/ml, about 4320 h*ng/ml, about 4500 h*ng/ml, about 4600 h*ng/ml, about 4700 h*ng/ml, or about 4772 h*ng/ml. In some embodiments, the oral solution comprising rofecoxib achieves a plasma AUC_{0-48 hr} of between 174.5 h*ng/ml and 276 h*ng/ml for each 1 mg of rofecoxib in the solution.

In some embodiments, the oral solution comprises rofecoxib and achieves a geometric mean plasma Cₘₐₓ from about 276 to about 432 ng/ml following administration of a single dose of the oral solution to a population of healthy adults less than 65 years of age. In some embodiments, the geometric mean plasma Cₘₐₓ is about 276 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, or about 432 ng/ml. In some embodiments, the oral solution comprising rofecoxib achieves a Cₘₐₓ of between 16 ng/ml and 25.1 ng/ml for each 1 mg of rofecoxib in the solution.

### BRIEF DESCRIPTION OF FIGURES

FIG. 1 shows results summary for screening vehicle compositions 169-203.

FIG. 2 shows results summary for screening vehicle compositions 204-219.

FIG. 3 shows results summary for screening vehicle compositions 135-168.

FIG. 4 shows Cmax values for the tablet and oral solution (OS) formulations of rofecoxib.

FIG. 5 shows AUC for the tablet and oral solution (OS) formulations of rofecoxib.

### DETAILED DESCRIPTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

As used herein, “solubilizing agent” refers to, but is not limited to, a cyclodextrin, a polyethylene glycol, a protic solvent, a dipolar aprotic solvent, a lipid, or a surfactant.

As used herein, “cyclodextrin” refers to a cyclic oligosaccharide. Cyclodextrin includes, but is not limited to, the compounds known as β-cyclodextrin, α-cyclodextrin, and γ-cyclodextrin, and derivatives thereof.

As used herein, a “water insoluble COX-2 inhibitor” includes any COX-2 inhibitor which is insoluble or practically insoluble in water. Water insoluble COX-2 inhibitor includes rofecoxib, etoricoxib, celecoxib, and pharmaceutically acceptable salts, esters, or co-crystals thereof.

As used herein, “treat,” “treating or “treatment” include alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition, and are intended to include prophylaxis.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, “administer,” “administering,” “administration,” refer to the methods used to enable delivery of a pharmaceutical compositions to the targeted location of biological action.

As used herein, “acceptable” with respect to a pharmaceutical composition refers to the composition having no persistent detrimental effect on the general health of a subject being treated.

As used herein, “antioxidant” refers to a pharmaceutical compound that prevents oxygen or oxygen-derived free radicals from interacting with other substances. The general function of antioxidants in compositions is to minimize or distort the oxidative processes that occur with some pharmaceutical compounds or excipients upon exposure to oxygen or in the presence of free radicals.

As used herein, “co-administration” means the administration of two agents (e.g. concomitantly or sequentially) in any manner in which the pharmacological effects of both are manifest in the subject at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

As used herein, the term “patient” refers to a human or animal.

As used herein, the term “mammal” includes, but is not limited to, a human, mouse, rat, guinea pig, dog, cat, horse, cow, pig, or non-human primate, such as a monkey, chimpanzee, baboon or rhesus. In one embodiment, the mammal is a human.

As used herein, “pain” refers to all types of pain, including, but not limited, to nociceptive pain, neuropathic pain, post-operative pain, lower back pain, cluster headaches, herpes neuralgia, phantom limb pain, central pain, dental pain, opioid-resistant pain, visceral pain, surgical pain, bone injury pain, pain during labor and delivery, pain resulting from burns, including sunburn, post-partum pain, migraine, and genitourinary tract-related pain including cystitis.

Non-Limiting Embodiments of the Compositions

In certain aspects, the subject matter disclosed herein provides a pharmaceutical composition comprising an aqueous solution comprising: a) water, b) a water insoluble COX-2 inhibitor or a pharmaceutically acceptable salt, ester or co-crystal thereof, and c) a solubilizing agent, wherein the solubility of the water insoluble COX-2 inhibitor in the solution is more than 10 μg/ml.

In some embodiments, the water insoluble COX-2 inhibitor is selected from the group consisting of rofecoxib, etoricoxib, and celecoxib. In some embodiments, the water insoluble COX-2 inhibitor is rofecoxib. In some embodiments, the composition further comprises at least one co-solvent helper. In some embodiments, the composition further comprises at least one antioxidant. In some embodiments, the composition further comprises at least one buffering agent. In some embodiments, the composition further comprises at least one isotonic agent.

In some embodiments, said composition is a reconstituted lyophile. In some embodiments, said pharmaceutical composition is diluted prior to administration.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the β-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the effective amount of the water insoluble COX-2 inhibitor in a single dose formulation is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the concentration % (w/v) of the water insoluble COX-2 inhibitor in the solution is selected from the group consisting of about 0.001%, about 0.002%, about 0.003%, about 0.004%, about 0.005%, about 0.006%, about 0.007%, about 0.008%, about 0.01%, about 0.012%, about 0.015%, about 0.017%, about 0.02%, about 0.025%, about 0.03%, about 0.035%, about 0.04%, about 0.05%, about 0.06%, about 0.07% and any other suitable concentration. In some embodiments, the volume of the solution is selected from the group consisting of about 5 mL, about 10 ml, about 20 ml, about 25 mL, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 180 ml, and any suitable volume. In some embodiments, the solubility of the water insoluble COX-2 inhibitor in the solution is more than 20 μg/ml, more than 30 μg/ml, more than 40 μg/ml, more than 50 μg/ml, more than 60 μg/ml, more than 70 μg/ml, more than 80 μg/ml, more than 90 μg/ml, more than 100 μg/ml, more than 110 μg/ml, more than 120 μg/ml, more than 130 μg/ml, more than 140 μg/ml, or more than 150 μg/ml.

In some embodiments, said composition is administered as part of a combination therapy with at least one other therapeutic agent. In some embodiments, said composition is suitable for intravenous administration to a subject.

In certain aspects, the subject matter disclosed herein provides a lyophilized pharmaceutical composition comprising a lyophilization product of any of the pharmaceutical compositions described herein.

In certain aspects, the subject matter disclosed herein provides a method for treatment of pain, fever, or inflammation in a subject in need thereof, the method comprising parenterally administering to the subject a therapeutically effective amount of a pharmaceutical composition comprising an aqueous solution comprising: a) water, b) a COX-2 inhibitor or a pharmaceutically acceptable salt or ester thereof, and c) a solubilizing agent.

In some embodiments, the water insoluble COX-2 inhibitor is selected from the group consisting of rofecoxib, etoricoxib, and celecoxib. In some embodiments, the water insoluble COX-2 inhibitor is rofecoxib.

In some embodiments, the composition further comprises at least one co-solvent helper. In some embodiments, the composition further comprises at least one antioxidant. In some embodiments, the composition further comprises at least one buffering agent. In some embodiments, the composition further comprises at least one isotonic agent. In some embodiments, said composition is a reconstituted lyophile. In some embodiments, said pharmaceutical composition is diluted.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the β-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the effective amount of the COX-2 inhibitor in a single dose formulation is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the concentration % (w/v) of the COX-2 inhibitor in an aqueous formulation is selected from the group consisting of about 0.001%, about 0.002%, about 0.003%, about 0.004%, about 0.005%, about 0.006%, about 0.007%, about 0.008%, about 0.01%, about 0.012%, about 0.015%, about 0.017%, about 0.02%, about 0.025%, about 0.03%, about 0.035%, about 0.04%, about 0.05%, about 0.06%, about 0.07% and any other suitable concentration. In some embodiments, the volume of an aqueous formulations of the COX-2 inhibitor is selected from the group consisting of about 1 ml, about 2 ml, about 3 ml, about 4 ml, about 5 ml, about 10 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 180 ml, and any suitable volume. In some embodiments, the solubility of the COX-2 inhibitor is enhanced to more than 10 μg/ml, more than 20 μg/ml, more than 30 μg/ml, more than 40 μg/ml, more than 50 μg/ml, more than 60 μg/ml, more than 70 μg/ml, more than 80 μg/ml, more than 90 μg/ml, more than 100 μg/ml, more than 110 μg/ml, more than 120 μg/ml, more than 130 μg/ml, more than 140 μg/ml, or more than 150 μg/ml. In some embodiments, said composition is administered as part of a combination therapy with at least one other therapeutic agent.

In certain aspects, the subject matter disclosed herein provides a lyophilized pharmaceutical composition comprising a water insoluble COX-2 inhibitor or a pharmaceutically acceptable salt, ester or co-crystal thereof and a solubilizing agent, wherein the lyophilized pharmaceutical composition is a produced by lyophilizing a pharmaceutical composition comprising an aqueous formulation comprising water, a water insoluble COX-2 inhibitor or a pharmaceutically acceptable salt, ester or co-crystal thereof, and a solubilizing agent.

In certain aspects, the subject matter disclosed herein provides a pharmaceutical composition comprising an oral solution comprising: a) a COX-2 inhibitor or a pharmaceutically acceptable salt, ester or co-crystal thereof, and b) a solubilizing agent. In some embodiments, the COX-2 inhibitor is a water insoluble COX-2 inhibitor.

In some embodiments, the COX-2 inhibitor is selected from the group consisting of rofecoxib, etoricoxib, and celecoxib. In some embodiments, the COX-2 inhibitor is rofecoxib. In some embodiments, the composition further comprises at least one co-solvent helper. In some embodiments, the composition further comprises at least one antioxidant. In some embodiments, the composition further comprises at least one buffering agent. In some embodiments, the pharmaceutical composition is diluted prior to administration.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the 3-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the pharmaceutical composition comprises an effective amount of the COX-2 inhibitor in a single dose, wherein the effective amount is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the pharmaceutical composition has a volume of the oral solution selected from the group consisting of about 5 ml, about 10 ml, about 15 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 165 ml, about 170 ml, about 175 ml, about 180 ml, about 185 ml, about 190 ml, about 200 ml, or any suitable volume. In some embodiments, the pharmaceutical composition the oral solution comprises of about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about 0.14 mg/ml, or about 0.15 mg/ml of rofecoxib.

In some embodiments, the pharmaceutical composition is administered as part of a combination therapy with at least one other therapeutic agent. In some embodiments, the pharmaceutical composition is suitable for oral administration to a subject.

In some embodiments, the oral solution comprises rofecoxib and achieves a geometric mean plasma AUC_{0-48 hr} from about 3053 to about 4772 h*ng/ml following administration of a single dose of the oral solution to a population of healthy adults less than 65 years of age. In some embodiments, the geometric mean plasma AUC_{0-48 hr} is about 3053 h*ng/ml, about 3100 h*ng/ml, about 3200 h*ng/ml, about 3300 h*ng/ml, about 3400 h*ng/ml, about 3500 h*ng/ml, about 3600 h*ng/ml, about 3700 h*ng/ml, about 3800 h*ng/ml, about 3900 h*ng/ml, about 4000 h*ng/ml, about 4100 h*ng/ml, about 4200 h*ng/ml, about 4300 h*ng/ml, about 4320 h*ng/ml, about 4500 h*ng/ml, about 4600 h*ng/ml, about 4700 h*ng/ml, or about 4772 h*ng/ml. In some embodiments, the oral solution comprising rofecoxib achieves a plasma AUC_{0-48 hr} of between 174.5 h*ng/ml and 276 h*ng/ml for each 1 mg of rofecoxib in the solution.

In some embodiments, the oral solution comprises rofecoxib and achieves a geometric mean plasma Cₘₐₓ from about 276 to about 432 ng/ml following administration of a single dose of the oral solution to a population of healthy adults less than 65 years of age. In some embodiments, the geometric mean plasma Cₘₐₓ is about 276 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, or about 432 ng/ml. In some embodiments, the oral solution comprising rofecoxib achieves a Cₘₐₓ of between 16 ng/ml and 25.1 ng/ml for each 1 mg of rofecoxib in the solution.

In certain aspects, the subject matter disclosed herein provides a method for treatment of pain, fever, or inflammation in a subject in need thereof, the method comprising administering to the subject a therapeutically effective amount of a pharmaceutical composition comprising an oral solution comprising: a) a COX-2 inhibitor or a pharmaceutically acceptable salt, ester or co-crystal thereof, and b) a solubilizing agent. In some embodiments, the COX-2 inhibitor is a water insoluble COX-2 inhibitor.

In some embodiments, the COX-2 inhibitor is selected from the group consisting of rofecoxib, etoricoxib, and celecoxib. In some embodiments, the COX-2 inhibitor is rofecoxib. In some embodiments, the composition further comprises at least one co-solvent helper. In some embodiments, the composition further comprises at least one antioxidant. In some embodiments, the composition further comprises at least one buffering agent. In some embodiments, the pharmaceutical composition is diluted prior to administration.

In some embodiments, the solubilizing agent is a cyclodextrin. In some embodiments, the cyclodextrin is selected from the group consisting of α-cyclodextrins, β-cyclodextrins, γ-cyclodextrins, and any mixtures thereof. In some embodiments, the β-cyclodextrin is a hydroxypropyl-β-cyclodextrin corresponding to the CAS Registry Number 128446-35-5. In some embodiments, the hydroxypropyl-β-cyclodextrin is Cavasol®. In some embodiments, the 3-cyclodextrin is a sulfobutyl ether-β-cyclodextrin corresponding to the CAS Registry Number 182410-00-0. In some embodiments, the sulfobutyl ether-β-cyclodextrin is Captisol®.

In some embodiments, the method comprises an effective amount of the COX-2 inhibitor in a single dose selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg. In some embodiments, the oral solution has a volume selected from the group consisting of about 5 ml, about 10 ml, about 15 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 165 ml, about 170 ml, about 175 ml, about 180 ml, about 185 ml, about 190 ml, about 200 ml, or any suitable volume. In some embodiments, the oral solution comprises about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about 0.14 mg/ml, or about 0.15 mg/ml of rofecoxib. In some embodiments, the composition is administered as part of a combination therapy with at least one other therapeutic agent.

In some embodiments, the oral solution comprises rofecoxib and achieves a geometric mean plasma AUC_{0-48 hr} from about 3053 to about 4772 h*ng/ml following administration of a single dose of the oral solution to a population of healthy adults less than 65 years of age. In some embodiments, the geometric mean plasma AUC_{0-48 hr} is about 3053 h*ng/ml, about 3100 h*ng/ml, about 3200 h*ng/ml, about 3300 h*ng/ml, about 3400 h*ng/ml, about 3500 h*ng/ml, about 3600 h*ng/ml, about 3700 h*ng/ml, about 3800 h*ng/ml, about 3900 h*ng/ml, about 4000 h*ng/ml, about 4100 h*ng/ml, about 4200 h*ng/ml, about 4300 h*ng/ml, about 4320 h*ng/ml, about 4500 h*ng/ml, about 4600 h*ng/ml, about 4700 h*ng/ml, or about 4772 h*ng/ml. In some embodiments, the oral solution comprising rofecoxib achieves a plasma AUC_{0-48 hr} of between 174.5 h*ng/ml and 276 h*ng/ml for each 1 mg of rofecoxib in the solution.

In some embodiments, the oral solution comprises rofecoxib and achieves a geometric mean plasma Cₘₐₓ from about 276 to about 432 ng/ml following administration of a single dose of the oral solution to a population of healthy adults less than 65 years of age. In some embodiments, the geometric mean plasma Cₘₐₓ is about 276 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, or about 432 ng/ml. In some embodiments, the oral solution comprising rofecoxib achieves a Cₘₐₓ of between 16 ng/ml and 25.1 ng/ml for each 1 mg of rofecoxib in the solution.

Pharmaceutical Compositions

Described herein are aqueous formulations of water insoluble COX-2 inhibitors and methods of treatment or prevention of pain, fever, or inflammation, including postoperative pain, by administering a pharmaceutical composition including a water insoluble COX-2 inhibitor and one or more solubilizing agents. In some embodiments, the solubilizing agent is a cyclodextrin. In some embodiments, the COX-2 inhibitor is rofecoxib. In some embodiments, the COX-2 inhibitor is etoricoxib. In some embodiments, the COX-2 inhibitor is celecoxib.

In some embodiments, the pharmaceutical compositions described herein comprise an aqueous solution including water, a solubilizing agent, and a water insoluble COX-2 inhibitor. In some embodiments, the solubilizing agent is a cyclodextrin. In some embodiments, the solubilizing agent comprises a polyethylene glycol. Exemplary embodiments of polyethylene glycol include, but are not limited to, PEG300 and PEG400. In some embodiments, the solubilizing agent is Soluplus®. In some embodiments, the solubilizing agent is a protic solvent. In some embodiments, the solubilizing agent is a dipolar aprotic solvent, for example DMSO or DMF. In some embodiments, the solubilizing agent is a lipid. In some embodiments, the solubilizing agent is soybean oil. In some embodiments, the solubilizing agent is a surfactant, for example Kolliphor®, polysorbate 20 (PS 20), or polysorbate 80 (PS 80). In some embodiments, the solubilizing agent is a co-solvent helper such as ethanol. In some embodiments, pharmaceutical compositions may include more than one solubilizing agent. In one embodiment, the solubilizing agents include a cyclodextrin and a polyethylene glycol. In another embodiment, the solubilizing agents include a cyclodextrin and Soluplus®. In some embodiments, the pharmaceutical compositions are suitable for administration to a subject. In some embodiments, the pharmaceutical compositions are suitable for oral or parenteral administration to a human. In some embodiments, the pharmaceutical compositions are suitable for intravenous administration to a human.

In some embodiments, the pharmaceutical compositions described herein are in a dose unit that is “ready to use” for injection or other parenteral administration. In some embodiments, the pharmaceutical compositions described herein are diluted with a sterile solution prior to administration. In some embodiments, the pharmaceutical compositions described herein are formed by reconstituting a lyophilization product (e.g., a powder) with a sterile solution.

In some embodiments, the cyclodextrin is a cyclic oligosaccharide. In some embodiments, the cyclodextrin is a β-cyclodextrin or a derivative thereof. In some embodiments, the cyclodextrin is an α-cyclodextrin or a derivative thereof. In some embodiments, the cyclodextrin is a γ-cyclodextrin or a derivative thereof.

In some embodiments, the β-cyclodextrin derivative is a hydroxypropyl-β-cyclodextrin corresponding to the CAS Registry Number 128446-35-5. In some embodiments, the hydroxypropyl-β-cyclodextrin is Cavasol®. In some embodiments, the β-cyclodextrin derivative is a sulfobutyl ether-β-cyclodextrin corresponding to the CAS Registry Number 182410-00-0. In some embodiments, the sulfobutyl ether-β-cyclodextrin is Captisol®.

In some embodiments, the “water insoluble COX-2 inhibitor” is a COX-2 inhibitor which is insoluble or practically insoluble in water. In some embodiments the water insoluble COX-2 inhibitor is rofecoxib. In some embodiments the water insoluble COX-2 inhibitor is etoricoxib. In some embodiments the water insoluble COX-2 inhibitor is celecoxib. In some embodiments the water insoluble COX-2 inhibitor has a solubility of less than 10 μg/ml.

In some embodiments, the total amount of the one or more solubilizing agent in the aqueous solution is about 5% (w/v), 10% (w/v), 15% (w/v), 20% (w/v), 25% (w/v), 30% (w/v), 35% (w/v), 40% (w/v/), 45% (w/v), 50% (w/v), 55% (w/v), 60% (w/v), 70% (w/v), 80% (w/v) or more. In some embodiments, the total amount of solubilizing agent(s) in the aqueous solution is about 5% (v/v), 10% (v/v), 15% (w/v), 20% (v/v), 25% (v/v), 30% (v/v), 35% (v/v), 40% (v/v/), 45% (v/v), 50% (v/v), 55% (v/v), 60% (v/v), 70% (w/v), 80% (v/v) or more.

In some embodiments, the total amount of the solubilizing agent(s) in the aqueous formulation ranges from 5-10% (w/v), 10-15% (w/v), 15-20% (w/v), 20-25% (w/v), 25-30% (w/v), 30-35% (w/v), 35-40% (w/v), 40-45% (w/v), 45-50% (w/v), 50-55% (w/v), or 55-60% (w/v), or 60-80% (w/v). In some embodiments, the total amount of solubilizing agent(s) in the aqueous formulation ranges from 5-10% (v/v), 10-15% (v/v), 15-20% (v/v), 20-25% (v/v), 25-30% (v/v), 30-35% (v/v), 35-40% (v/v), 40-45% (v/v), 45-50% (v/v), 50-55% (v/v), 55-60% (v/v), or 60-80% (v/v).

In some embodiments, the subject matter described herein relates to enhanced solubility of water insoluble COX-2 inhibitors. In some embodiments, the solubility of COX-2 inhibitors is enhanced to more than 10 μg/ml, more than 20 μg/ml, more than 30 μg/ml, more than 40 μg/ml, more than 50 μg/ml, more than 60 μg/ml, more than 70 μg/ml, more than 80 μg/ml, more than 90 μg/ml, more than 100 μg/ml, more than 110 μg/ml, more than 120 μg/ml, more than 130 μg/ml, more than 140 μg/ml, or more than 150 μg/ml. In some embodiments, the solubility of the water insoluble COX-2 inhibitors is measured at 4 hours. In some embodiments, the solubility of the water insoluble COX-2 inhibitors is measured at 24 hours. In some embodiments, the solubility of the water insoluble COX-2 inhibitors is measured at 48 hours.

In some embodiments, the pharmaceutical compositions described herein include one or more viscosity modifiers. In some embodiments, the viscosity modifier is benzyl alcohol.

Antioxidants

In some embodiments, the pharmaceutical composition described herein also contains at least one antioxidant, which may increase the stability of the COX-2 inhibitor in solution. Examples of suitable antioxidants include, but are not limited to, citric acid monohydrate, histidine, monothioglycerol, niacinamide, phosphoric acid, potassium metabisulfite, sodium ascorbate, sodium bisulfate acetone, sodium formaldehyde sulfoxylate, sodium metabisulfite, sodium thiosulfate, tartaric acid, or any derivative or combination thereof. Other examples of suitable antioxidants include, but are not limited to, cysteine hydrochloride monohydrate, thiolyglycolic acid, thiolacetic acid, dithiothreitol, reduced glutathione, thiourea, alpha-thioglycerol, cysteine, acetylcysteine, methionine, mercaptoethane, sulfonic acid, metabisulfite, ascorbic acid ascorbic acid derivatives (e.g., ascorbyl palmitate), sodium citrate, an organic compound having at least one thiol, an alkyl polyhydroxylated compound, a cycloalkyl polyhydroxylated compound, a hydroxypolycarboxylic acid, an alpha-hydroxy polycarboxylic acid (e.g., citric acid), tocotrienol, dimethyl glycine, betaine, butylated hydroxyanisole, butylated hydroxytoluene, tocopherol, polyethylene glycol, succinate, butylated hydroxytoluene (BHT), butylated hydroxyanisole (BHA), propyl gallate, hydroquinone, hydroxycoumarins, ethanolamine, lecithin, cephalin, malic acid, sorbitol, phosphoric acid, thiodipropionic acid and its esters, dithiocarbamates or any combination thereof. In some embodiments, the pharmaceutical composition is free of polyethylene glycol or a derivative thereof. In other embodiments, the pharmaceutical composition is free of sulfites. In some embodiments, the antioxidant is cysteine hydrochloride monohydrate. In some embodiments, the antioxidant is mannitol.

In some embodiments, the antioxidant is any antioxidant that can be used with the pharmaceutical compositions described herein. In some embodiments, the antioxidant is in a solution form before it is incorporated into a pharmaceutical composition. In some embodiments, the antioxidant solution includes ethanol. In some embodiments, the antioxidant solution may be the same as a solubilizing agent, such as ethanol. In some embodiments, the antioxidant helps prevent formation of an oxidation product of rofecoxib, including 4-[4-(methylsulfonyl)phenyl]-3-phenyl-2,5-furandione, 4-[4-(methylsulfinyl)phenyl]-3-phenyl-2(5H)-furanone, and/or dicarboxylate derivative of rofecoxib. In some embodiments, the antioxidant reduces the formation of 4-[4-(methylsulfonyl)phenyl]-3-phenyl-2,5-furandione, 4-[4-(methylsulfinyl)phenyl]-3-phenyl-2(5H)-furanone, and/or dicarboxylate derivative of rofecoxib by 5%, 10%, 15%, 20%, 25%, 50%, or 75% or more.

In some embodiments, the amount % (w/w) of the antioxidant in a solid or lyophilized formulation of the water insoluble COX-2 inhibitor prior to reconstitution is about 0.01% (w/w) to about 5.0% (w/w). In some embodiments, the amount % (w/w) of the antioxidant is about 0.01% (w/w), 0.05% (w/w), 0.10% (w/w), 0.15% (w/w), about 0.17% (w/w), about 0.20% (w/w), about 0.30% (w/w), about 0.40% (w/w), about 0.45% (w/w), about 0.50% (w/w), about 0.52% (w/w), about 0.55% (w/w), about 0.60% (w/w), about 0.70% (w/w), about 0.80% (w/w), about 1.0% (w/w), about 1.3% (w/w), about 1.5% (w/w), about 1.7% (w/w), about 2.0% (w/w), about 2.2% (w/w), about 2.3% (w/w), about 2.5% (w/w), about 2.7% (w/w), about 2.8% (w/w), about 3.0% (w/w), about 3.2% (w/w), about 3.5% (w/w), about 3.6% (w/w), about 4.0% (w/w), about 4.7% (w/w), or any other suitable amount of antioxidant % (w/w) from about 0.10% (w/w) to about 5.0% (w/w). In some embodiments, the amount % (w/w) of antioxidant is about 0.30% (w/w) to about 1.0% (w/w). In some embodiments, the amount % (w/w) of antioxidant is about 0.50% (w/w).

In some embodiments, the concentration of the antioxidant in an aqueous formulation of a water insoluble COX-2 inhibitor prior to administration ranges from about 0.01 mg/ml to about 10 mg/ml. In some embodiments, the concentration of the antioxidant is about 0.02 mg/ml, about 0.03 mg/ml, about 0.05 mg/ml, about 0.08 mg/ml, about 0.09 mg/ml, about 0.10 mg/ml, about 0.12 mg/ml, about 0.13 mg/ml, about 0.15 mg/ml, about 0.18 mg/ml, about 0.20 mg/ml, about 0.22 mg/ml, about 0.25 mg/ml, about 0.27 mg/ml, about 0.30 mg/ml, about 0.40 mg/ml, about 0.45 mg/ml, about 0.50 mg/ml, about 0.60 mg/ml, about 0.80 mg/ml, about 1.2 mg/ml, about 1.5 mg/ml, about 2.0 mg/ml, about 2.5 mg/ml, about 3.0 mg/ml, about 3.5 mg/ml, about 4.0 mg/ml, about 5.0 mg/ml, about 6.0 mg/ml, about 7.5 mg/ml, about 8.0 mg/ml, about 9 mg/ml, about 9.5 mg/ml, or any other suitable concentration of antioxidant from about 0.01 mg/ml to about 10 mg/ml. In some embodiments, the concentration of the antioxidant is about 0.08 mg/ml to about 0.50 mg/ml. In some embodiments, the concentration of the antioxidant is about 0.25 mg/ml.

Buffering Agents

In some embodiments, an aqueous formulation of a COX-2 inhibitor contains at least one buffering agent, which may maintain the pH of the formulation within an acceptable range as described herein. In some embodiments, the buffer used is a buffer compatible with parenteral administration in a subject, the pH of which may be adjusted between about 2 and about 8. In some embodiments, the pH of an aqueous formulation of a COX-2 inhibitor is from about pH 2 to about pH 8, pH 3 to about pH 8, or pH 4 to about pH 8. In some embodiments, the pH is about pH 4.5, about pH 4.6, about pH 4.8, about pH 5.0, about pH 5.5, about pH 6.2, about pH 6.5, about pH 7.5, or any other suitable pH value from about pH4 to about pH 8. In some embodiments, the pH of the aqueous formulation is from about pH 5 to about pH 7.0. In some embodiments, the pH is about pH 5.2, about pH 5.5, about pH 5.6, about pH 6.0, about pH 6.4, or any other suitable pH value from about pH 5 to about pH 7.0. In some embodiments, the aqueous formulation of a COX-2 inhibitor has a pH of about 5 to about 6.

In some embodiments, an aqueous formulation of a COX-2 inhibitor contains at least one buffering agent with a pKa from about 4.5 to about 6.5. In some embodiments, the pKa is about 4.6, about 4.8, about 5.0, about 5.2, about 5.3, about 5.4, about 5.5, about 5.8, about 6.0, about 6.2, about 6.4, or any other suitable pKa from about 4.5 to about 6.5.

In some embodiments, the buffering agent is a pharmaceutically acceptable salt or acid of citrate, phosphate, acetate, glutamate, tartrate, benzoate, lactate, histidine or other amino acids, gluconate, malate, tryptophan, succinate, formate, propionate, carbonate, or any combination thereof adjusted to an appropriate pH, as described herein, with acid (for example, hydrochloric acid) or base (for example, sodium hydroxide).

In some embodiments, the amount % (w/w) of the buffering agent in the solid or lyophilized formulation of the COX-2 inhibitor prior to reconstitution is about 0.05% (w/w) to about 2% (w/w). In some embodiment, the amount % (w/w) of the buffering agent lyophilized form is about 0.08% (w/w), about 0.10% (w/w), about 0.15% (w/w), about 1.0% (w/w), about 1.3% (w/w), about 1.5% (w/w), about 1.7% (w/w), about 0.20% (w/w), about 0.22% (w/w), about 0.25% (w/w), about 0.26% (w/w), about 0.27% (w/w), about 0.28% (w/w), about 0.30% (w/w), about 0.35% (w/w), about 0.40% (w/w), about 0.50% (w/w), about 0.60% (w/w), about 0.70% (w/w), about 0.80% (w/w), about 1.2% (w/w), about 1.4% (w/w), about 1.5% (w/w), about 1.7%, or any other suitable amount of buffering agent % (w/w) from about 0.05% (w/w) to about 2.0% (w/w). In some embodiments, the amount % (w/w) of the buffering agent is about 0.10% to about 0.70%. In some embodiments, the amount % (w/w) of the buffering agent is about 0.26%.

In some embodiments, the concentration of the buffering agent in an aqueous formulation of a COX-2 inhibitor prior to administration ranges from about 0.01 mg/ml to about 10 mg/ml. In some embodiments the concertation is about 0.02 mg/ml, about 0.03 mg/ml, about 0.05 mg/ml, about 0.08 mg/ml, about 0.09 mg/ml, about 0.10 mg/ml, about 0.12 mg/ml, about 0.13 mg/ml, about 0.15 mg/ml, about 0.30 mg/ml, about 0.5 mg/ml, about 0.8 mg/ml, about 1.2 mg/ml, about 1.5 mg/ml, about 2.0 mg/ml, about 2.5 mg/ml, about 3.0 mg/ml, about 3.5 mg/ml, about 4.0 mg/ml, about 5.0 mg/ml, about 6.0 mg/ml, about 7.5 mg/ml, about 8.0 mg/ml, about 9 mg/ml, about 9.5 mg/ml, or any other suitable concentration of buffering agent from about 0.01 mg/ml to about 10 mg/ml. In some embodiments, the concentration of buffering agent is about 0.08 mg/ml to about 0.30 mg/ml. In some embodiments, the concentration of buffering agent is about 0.13 mg/ml.

In some embodiments, the buffering agent helps prevent formation of an oxidation product of rofecoxib, including 4-[4-(methylsulfonyl)phenyl]-3-phenyl-2,5-furandione and/or 4-[4-(methylsulfinyl)phenyl]-3-phenyl-2(5H)-furanone. In some embodiments, the buffering agent reduces the formation of 4-[4-(methylsulfonyl)phenyl]-3-phenyl-2,5-furandione and/or 4-[4-(methylsulfinyl)phenyl]-3-phenyl-2(5H)-furanone by 25%, 50%, or 75% or more.

Isotonicity Agents

In some embodiments, an aqueous formulation of a COX-2 inhibitor also contains one or more isotonicity agents, which may maintain the osmolality of the formulation in a range that is physiologically compatible. In some embodiments, the osmolality of the formulation is physiologically compatible with an intravenous administration of the inhibitor. In some embodiments, the osmolality of the formulation is about 230 mOsm/L to about 420 mOsm/L. In some embodiments, the osmolality is about 240 mOsm/L, about 250 mOsm/L, about 260 mOsm/L, about 270 mOsm/L, about 276 mOsm/L, about 290 mOsm/L, about 300 mOsm/L, about 305 mOsm/L, about 310 mOsm/L, about 320 mOsm/L, about 350 mOsm/L, about 375 mOsm/L, about 400 mOsm/L or any other suitable osmolality from about 240 mOsm/L to about 420 mOsm/L. In some embodiments, the osmolality of the aqueous formulation is about 276 mOsm/L to about 320 mOsm/L. In some embodiments, the osmolality of the formulation is about 290 mOsm/L, about 295 mOsm/L, about 300 mOsm/L, about 305 mOsm/L, about 310 mOsm/L, about 315 mOsm/L, or any other suitable osmolality from about 276 mOsm/L to about 320 mOsm/L. In one embodiment, the osmolality of the aqueous formulation is about 200 mOsm/L-400 mOsm/L. Suitable agents for adjusting the isotonicity of the aqueous formulations of COX-2 inhibitors include, but are not limited to, mannitol, sorbitol, glycerol, sucrose, glucose, dextrose, levulose, fructose, lactose, polyethylene glycols 400 to 4000, phosphates, sodium chloride, potassium chloride, calcium chloride, calcium glucono-glucoheptonate, dimethyl sulfone. In some embodiments, the isotonicity agent is mannitol. In some embodiments, the isotonic agent is sodium chloride.

In some embodiments, the amount % (w/w) of the isotonicity agent in the solid or lyophilized form of the COX-2 inhibitor formulation prior to reconstitution is about 5% (w/w) to about 95% (w/w). In some embodiments, the amount % (w/w) of the isotonicity agent is about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 72% (w/w), about 74% (w/w), about 76% (w/w), about 78% (w/w), about 79% (w/w), about 80% (w/w), about 81% (w/w), about 82% (w/w), about 84% (w/w), about 86% (w/w), about 90% (w/w), about 92% (w/w), or any other suitable amount of isotonicity agent % (w/w) from about 5% (w/w) to about 95% (w/w). In some embodiments, the amount of isotonicity agent % (w/w) is about 65% (w/w) to about 85% (w/w). In some embodiments, the amount of isotonicity agent % (w/w) is about 79%.

In some embodiments, the concentration of the isotonicity agent in an aqueous formulation of a COX-2 inhibitor prior to administration ranges from about 1.0 mg/ml to about 150 mg/ml. In some embodiments, the concentration of the isotonicity agent is about 1.0 mg/ml, about 2.0 mg/ml, about 3.0 mg/ml, about 3.5 mg/ml, about 4.0 mg/ml, about 4.5 mg/ml, about 5.0 mg/ml, about 8.0 mg/ml, about 12 mg/ml, about 15 mg/ml, about 20 mg/ml, about 25 mg/ml, about 30 mg/ml, about 32 mg/ml, about 35 mg/ml, about 37 mg/ml, about 38 mg/ml, about 40 mg/ml, about 50 mg/ml, about 60 mg/ml, about 75 mg/ml, about 80 mg/ml, about 90 mg/ml, about 95 mg/ml, about 100 mg/ml, about 110 mg/ml, about 120 mg/ml, about 140 mg/ml, or any other suitable concentration of isotonicity agent from about 5 mg/ml to about 150 mg/ml.

In some embodiments, the aqueous formulation does not include one or more of the following excipients: ethyl alcohol, glycerin, glyceryl monocaprylate, L-menthol, lauroyl polyoxyl-32 glycerides, medium chain triglycerides, monoammonium glycyrrhizinate, peppermint flavor, polyoxyl 35 castor oil, polyoxyl 40 hydrogenated castor oil, propyl gallate, or sucralose.

Combination Therapies

The pharmaceutical compositions described herein can also be administered in combination with other therapeutic reagents. In some embodiments, such additional therapeutic agents include, but are not limited to, analgesics, antipyretics, or anti-inflammatory agents. In some embodiments, where a combination therapy is employed, other agents do not have to be administered in the same pharmaceutical composition as the COX-2 inhibitor, and may, because of different physical and chemical characteristics, be administered by different routes.

In some embodiments, the therapeutic agent may be administered concurrently (for example, simultaneously, essentially simultaneously or within the same treatment protocol) or sequentially, depending upon the severity of pain experienced by the patient, the nature of the disease, disorder, or condition, the condition, and the actual choice of compounds used. For combination therapies described herein, dosages of the compounds to be co-administered with an aqueous formulation of the COX-2 inhibitor will vary depending on the type of co-drug employed, on the amount of pain experienced by the patient, the risk for addiction, the disease or condition being treated among other factors. In addition, when co-administered with one or more biologically active agents, the aqueous formulation of a COX-2 inhibitor provided herein may be administered either simultaneously with the biologically active agent(s), or sequentially.

The multiple therapeutic agents (one of which is the aqueous formulation of a COX-2 inhibitor as described herein) may be administered in any order or even simultaneously. If simultaneously, the multiple therapeutic agents may be provided in a single, unified intravenous form, or in multiple forms (by way of example only, either as a single intravenous formulation, as multiple intravenous formulations, or as intravenous formulation and a pill). One of the therapeutic agents may be given in multiple doses, or both may be given as multiple doses. If not simultaneous, the timing between the multiple doses may vary from more than 1 minute to less than 12 hours. In some embodiments, the timing between the multiple doses is from between about 1 minute to about 6 hours, or about 1 minute and about 3 hours, or about 1 minute and about 1 hour. In addition, the combination methods, compositions and formulations are not to be limited to the use of only two agents; the use of multiple therapeutic combinations is also envisioned.

The pharmaceutical agents which make up the combination therapy disclosed herein may be a combined dosage form (i.e., a combined IV formulation) or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from a few minutes to several hours, depending upon the properties of each pharmaceutical agent, such as potency, solubility, bioavailability, plasma half-life and kinetic profile of the pharmaceutical agent.

The compounds described herein and combination therapies can be administered before, during or after the occurrence of a fever or painful condition, and the timing of administering the composition containing a compound can vary. Thus, for example, the compounds can be used as a prophylactic and can be administered continuously to subjects with a propensity to develop conditions (e.g., body aches and chills following chemotherapy treatment) or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms.

Formulations

Pharmaceutical Composition for Intravenous Administration:

In some embodiments, intravenous formulations of the pharmaceutical compositions described herein are in the form of a powder to be reconstituted in solution under sterile conditions prior to administration. In some embodiments, the powder is a lyophilization product of a solution described herein. In other embodiments, the intravenous formulations are provided as sterile solutions ready for administration. In other embodiments, the intravenous formulations are in a concentrated form and ready for administration following dilution with a solution. Appropriate containers for storage and transport of the pharmaceutical compositions described herein include, but are not limited to, vials, bags, bottles, ampules, or any suitable containers for intravenous formulations known in the art.

“Ready to Use” Pharmaceutical Composition

In some embodiments, the pharmaceutical compositions disclosed herein can be prepared and administered in dose units. Liquid dose units are vials or ampoules for injection or other parenteral administration. In some embodiments, the vial or ampule contains 50 ml, 60 ml, 70 ml, 80, ml, 90 ml, 100 ml, 110 ml, 120 ml, 130 ml, 140 ml, 150 ml of a COX-2 inhibitor solution. In some embodiments the vial or ampule contains 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg or more of a water insoluble COX-2 inhibitor.

“Concentrate” Pharmaceutical Composition

In some embodiments, the pharmaceutical composition described herein is diluted with a water-based solution to create an infusate that is ready for administration. Several types of concentrated compositions can be used. The concentrate can contain 50 μg/ml, 100 μg/ml, 150 μg/ml, 200 μg/ml, 250 μg/ml, 300 μg/ml, 350 μg/ml, 400 μg/ml, 450 μg/ml, 500 μg/ml or more of the water insoluble COX-2 inhibitor. In one embodiment, a bag, vial, bottle, or other container is partially filled with the concentrated pharmaceutical composition and a pharmacist or healthcare provide adds 10 ml, 20 ml, 30 ml, 40 ml, 50 ml, 60 ml, 70 ml, 80 ml, 90 ml, 100 ml, 110 ml, 120 ml, 130 ml, 140 ml, 150 ml, 160 ml, 170 ml, 180 ml, 190 ml, 200 ml or more of a diluent to the container prior to intravenous administration. In another embodiment, a bag, vial, bottle, or other container with the concentrated pharmaceutical composition is provided and a pharmacist or healthcare provider removes 10 ml, 20 ml, 30 ml, 40 ml, 50 ml, 60 ml, 70 ml, 80 ml, 90 ml, 100 ml, 110 ml, 120 ml, 130 ml, 140 ml, 150 ml, 160 ml, 170 ml, 180 ml, 190 ml, or 200 ml of the concentrate and adds it to 10 ml, 20 ml, 30 ml, 40 ml, 50 ml, 60 ml, 70 ml, 80 ml, 90 ml, 100 ml, 110 ml, 120 ml, 130 ml, 140 ml, 150 ml, 160 ml, 170 ml, 180 ml, 190 ml, 200 ml or more of a diluent to create the infusate. In yet another embodiment, an empty bag, vial, bottle, or other container is provided and a pharmacist or healthcare provider adds 10 ml, 20 ml, 30 ml, 40 ml, 50 ml, 60 ml, 70 ml, 80 ml, 90 ml, 100 ml or more of diluent and 5 ml, 10 ml, 20 ml, 30 ml, 40 ml, 50 ml, 60 ml, 70 ml, 80 ml, 90 ml, 100 ml or more of concentrate to create an infusate. In some embodiments, the infusate comprises 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, 100 ml or more of the concentrate and diluent.

“Lyophilized” Pharmaceutical Composition

In some embodiments, the pharmaceutical composition described herein may be a lyophilized pharmaceutical composition comprising a lyophilization product of the aqueous formulations described herein. The lyophilization product may comprise 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg or more of a water insoluble COX-2 inhibitor. The lyophilization product may be reconstituted in solution under sterile conditions prior to administration.

In some embodiments, the pharmaceutical composition is administered intravenously. In some embodiments, the pharmaceutical composition is an oral suspension. In some embodiments, the pharmaceutical composition is an oral solution. In some embodiments, the pharmaceutical composition is a solution suitable for injection.

Pharmaceutical Composition for Oral Administration:

In some embodiments, the oral solution may further contain at least one flavoring agent or taste masking agent. Non-limiting exemplary list of flavoring agents/taste masking agents: natural and synthetic flavoring liquids such as volatile oils, synthetic flavor oils, flavoring aromatic oils, liquids, oleoresins or extracts derived from plants, leaves, flowers, fruits, stems and combinations thereof. Non-limiting representative examples of volatile oils include spearmint oil, cinnamon oil, oil of wintergreen (methyl salicylate), peppermint oil, menthol, clove oil, bay oil, anise oil, eucalyptus oil, thyme oil, cedar leaf oil, oil of nutmeg, allspice oil, oil of sage, mace extract, oil of bitter almond, and cassia oil. Also artificial, natural or synthetic flavors including fruit flavors such as vanilla, and citrus oils including lemon, orange, grape, lime and grapefruit and fruit essences including apple, pear, peach, grape, strawberry, raspberry, cherry, plum, pineapple, apricot, banana and other useful flavorings include aldehydes and esters such as benzaldehyde (cherry, almond), citral, i.e., alphocitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), bubble gum flavor, mixtures thereof and the like.

In some embodiments, the oral solution may further contain a sweetener. Non-limiting exemplary list of sweeteners: sugars such as sucrose, glucose (corn syrup), dextrose, invert sugar, fructose, and mixtures thereof, saccharin and its various salts such as the sodium or calcium salt; cyclamic acid and its various salts such as the sodium salt; the dipeptide sweeteners such as aspartame, acesulfame K, and other sweeteners like magnasweet, sucralose, mixtures thereof and the like.

Methods

In some embodiments, the subject matter disclosed herein provides a method of treating pain, fever, or inflammation in a subject in need thereof, wherein a pharmaceutical composition including a water insoluble COX-2 inhibitor is administered to the subject. In some embodiments, the subject matter disclosed herein provides a method of managing mild to moderate pain in a subject, wherein a pharmaceutical composition including a water insoluble COX-2 inhibitor is administered to the subject. In some embodiments, the subject matter disclosed herein provides a method of managing moderate to severe pain, wherein a pharmaceutical composition including a water insoluble COX-2 inhibitor is administered to the subject with adjunctive opioid analgesics. In some embodiments, the subject matter disclosed herein provides a method of reducing fever in a subject, wherein a pharmaceutical composition including a water insoluble COX-2 inhibitor is administered to the subject. In some embodiments, the subject matter disclosed herein provides a method of short-term (<5 days) management of moderately severe acute pain, which requires analgesia at the opioid level in a subject, wherein a pharmaceutical composition including a water insoluble COX-2 inhibitor is administered to the subject.

The aqueous formulations of the water insoluble COX-2 inhibitors described herein can be used for reducing pain conditions including, but not limited to, acute nociceptive pain, acute neuropathic pain, postoperative pain, lower back pain, cluster headaches, herpes neuralgia, phantom limb pain, central pain, dental pain, opioid-resistant pain, visceral pain, surgical pain, procedural pain, bone injury pain, pain during labor and delivery, pain resulting from burns, post-partum pain, headache, muscular aches, backache, arthritis pain, the common cold, toothache, dental pain, osteoarthritis pain, menstrual pain, menstrual cramps, migraine, and genitourinary tract-related pain including cystitis. In some embodiments, the aqueous formulation is administered prior to the onset of pain or a pain inducing condition or stimulus, for example prior to a surgical operation. In some embodiments, the aqueous formulations of water insoluble COX-2 inhibitors described herein are used to reduce fever, including, but not limited to, fever due to infections, drug reactions, allergic reactions, transfusion reactions, stroke, surgery, heat stroke, rheumatic diseases, cancer, or fever of unknown origin. In some embodiments, the aqueous formulations described herein are administered to a patient undergoing a dental procedure.

Dosages and Dosing Regimens

In some embodiments a single dose formulation of a water insoluble COX-2 inhibitor contains 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg.

In some embodiments, the concentration of a water insoluble COX-2 inhibitor in an aqueous formulation is about 0.001% (w/v) to about 0.07% (w/v). In some embodiments, the concentration (w/v) of a COX-2 inhibitor in an aqueous formulation is about 0.001%, about 0.002%, about 0.003%, about 0.004%, about 0.005%, about 0.006%, about 0.007%, about 0.008%, about 0.01%, about 0.012%, about 0.015%, about 0.017%, about 0.02%, about 0.025%, about 0.03%, about 0.035%, about 0.04%, about 0.05%, about 0.06%, about 0.07% or any other suitable concentration. In one embodiment, the concentration of the COX-2 inhibitor is from about 0.01% to about 0.03% (w/v).

Depending on the concentration of the water insoluble COX-2 inhibitor in the aqueous formulation and consistent with the suitable dose levels described herein, the volume of aqueous formulation administered can vary from about 1 ml to about 200 ml. In some embodiments, the volume of an aqueous formulation is about 1 ml, 2 ml, 3 ml, 4 ml, 5 ml, 10 ml, 20 ml, 25 mL, 30 ml, 40 ml, 50 ml, 60 ml, 70 ml, 80, ml, 90 ml, 100 ml, 110 ml, 120 ml, 130 ml, 140 ml, 150 ml, 160 ml, 180 ml, or any suitable volume of the aqueous formulation. In some embodiments, the volume of the aqueous formulation is about 75 ml to about 125 ml. In another embodiment, the volume is about 40 ml to about 75 ml. In yet another embodiment, the volume of the aqueous formulation is about 100 ml.

In some embodiments, the dosing regimen of the pharmaceutical composition described herein is a once daily administration. In some embodiments, the dosing regimen of the pharmaceutical composition described herein is multiple times per day. In some embodiment, the dosing regimen of the pharmaceutical composition described herein is twice per day, three times per day, four times per day, five times per day, or six times per day. In some embodiments, the dosing regimen is three times per day of intravenous administration of the pharmaceutical composition. In some embodiments, the dosing regimen is four times per day of intravenous administration of the pharmaceutical composition. In some embodiments, the pharmaceutical composition comprises 5 mg to 15 mg of rofecoxib and is administered in a 100 ml infusate three to four times per day.

An aqueous formulation of a water insoluble COX-2 inhibitor can be administered in an interval to allow for the administration of about 10 mg to about 200 mg in a 24 hour period. In some embodiments, the aqueous formulation is administered in an interval sufficient to allow for the administration of about 20 mg to 100 mg in a 24 hour period. In some embodiments, the aqueous formulation is administered between 1 to 6 times every twenty-four hours. In some embodiments, the frequency of administration is not greater than once every four hours.

In various embodiments, the aqueous formulation of a water insoluble COX-2 inhibitor is dosed so as to provide less than about 20 mg, 40 mg, 60 mg, 80 mg, 100 mg, 120 mg, 140 mg, 160 mg, 180 mg, or 200 mg over a 24-hour period. In various embodiments, the aqueous formulation is dosed three to six times in a 24-hour period. For example, in some embodiments, the aqueous formulation is dosed three times in a 24-hour period. In other embodiments, the aqueous formulation is dosed four times in a 24-hour period. In still other embodiments, the aqueous formulation is dosed five times in a 24-hour period. In some embodiments, the aqueous formulation is dosed six times in a 24-hour period. In some embodiments, the aqueous formulation is dosed seven times in a 24-hour period. In some embodiments, the aqueous formulation is dosed eight times in a 24-hour period.

In some embodiments, the infusion time of the intravenous formulation of the COX-2 inhibitor is about 10 min, about 15 min, about 20 min, about 25 min, about 30 min, about 40 min, about 45 min, about 50 min, about 60 min or more. In some embodiments, the infusion time is between 10 min and 30 min. In some embodiments, the infusion time ranges from about 1 minute to about 1 hour. In some embodiments, the infusion time is about 5 minutes, about 10 minutes, about 11 minutes, about 15 minutes, about 20 minutes, about 30 minutes, about 45 minutes, or any other suitable administration time from about 1 minute to about 1 hour. In some embodiments, the amount of time required for administration of the intravenous formulation ranges from about 5 minutes to about 45 minutes, or about 5 minutes to about 30 minutes, or about 5 minutes to about 15 minutes.

In some embodiments, the aqueous formulation of a COX-2 inhibitor is administered to a subject within about 12 hours after a surgical intervention, within 11 hours, 10 hours, 9 hours, 8 hours, 6 hours, 5 hours, 4 hours, 3 hours, 2 hours, 1 hours, 45 minutes, 30 minutes 15 minutes, 5 minutes, or any period within about 12 hours following a surgical intervention. In some embodiments, a subject is administered the aqueous formulation prior to a surgical intervention, about 4 hours or less prior to the surgical intervention, about 3 hours, 2 hours, 1 hours, 30 minutes, 15 minutes or during the surgical intervention itself.

### EXAMPLES

### Example 1

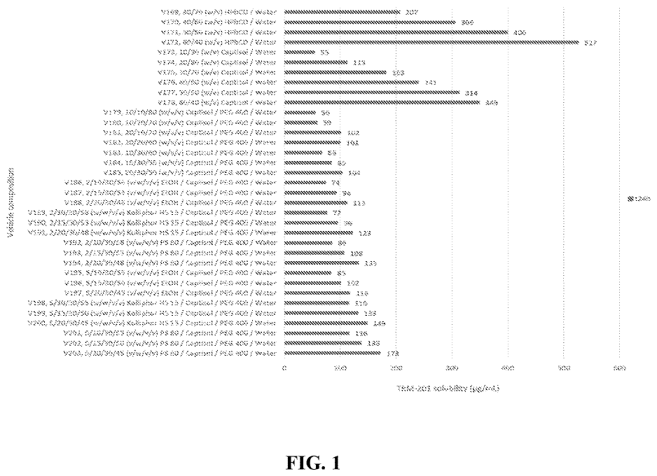

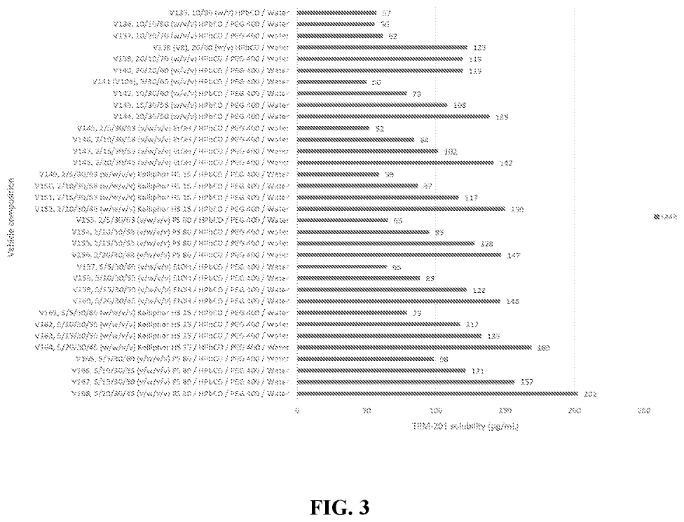

In some embodiments, the compositions described herein include the COX-2 inhibitor rofecoxib (also known as TRM-201) in a solution comprising hydroxypropyl β-cyclodextrin (HPbCD) or sulfobutyl ether-β-cyclodextrin (e.g., Captisol®) and water as shown in FIGS. 1 and 3 and Table 1 below:

TABLE 1
Vehicle Compositions.

| | | TRM-201 solubility |
|---|---|---|
| Vehicle # | Vehicle Composition | (μg/mL) @ t = 24 h |
| V138 (V8) | 20/80 (w/v) HPbCD / Water | 123 |
| V169 | 30/70 (w/v) HPbCD / Water | 207 |
| V170 | 40/60 (w/v) HPbCD / Water | 306 |
| V171 | 50/50 (w/v) HPbCD / Water | 400 |
| V172 | 60/40 (w/v) HPbCD / Water | 527 |
| V174 | 20/80 (w/v) Captisol ® / Water | 113 |
| V175 | 30/70 (w/v) Captisol ® / Water | 183 |
| V176 | 40/60 (w/v) Captisol ® / Water | 241 |
| V177 | 50/50 (w/v) Captisol ® / Water | 314 |
| V178 | 60/40 (w/v) Captisol ® / Water | 349 |

By comparison, the prior art, U.S. Pat. No. 6,063,811, teaches an intravenous formulation of rofecoxib comprising only 1 mg of rofecoxib per 200 mL of solution.

### Example 2

In some embodiments, the vehicle composition includes a cyclodextrin and water in a 15/85 (w/v) ratio. In some embodiments, addition of a second solubilizing agent to the vehicle composition increases solubility of the water insoluble COX-2 inhibitor. In some embodiments, addition of a second solubilizing agent increases the solubility of the water insoluble COX-2 inhibitor to 110 μg/ml, 115 μg/ml, 120 μg/ml, 130 μg/ml, 140 μg/ml or greater.

In some embodiments, the second solubilizing agent is a co-solvent helper such as ethanol, PS 20, or PS 80. In some embodiments, the second solubilizing agent is Soluplus®. In some embodiments, addition of the second solubilizing agent increases the solubility of the water insoluble COX-2 inhibitor to 120 μg/ml or greater.

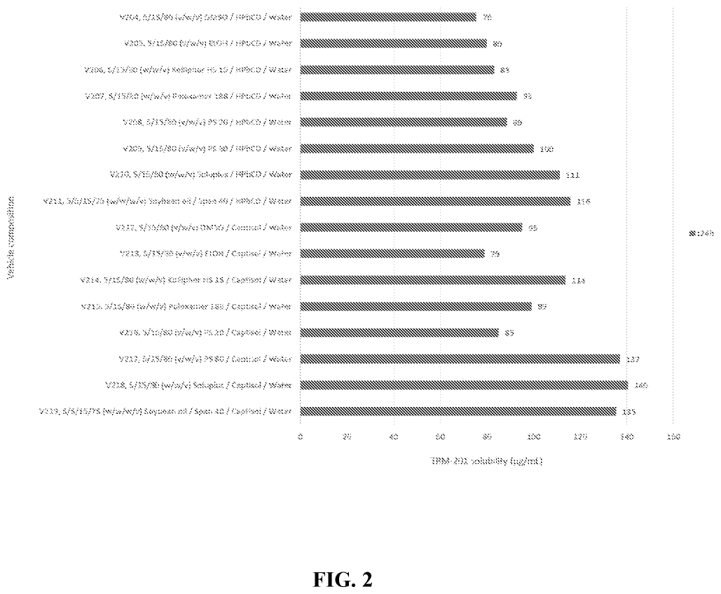

A study was performed to assess the solubility of rofecoxib in a composition comprising water, a cyclodextrin, and a second solubilizing agent. 20 mg of TRM-201 (rofecoxib) was mixed with 1.2 ml of each vehicle solution in a 2-ml microcentrifuge tube and tumbled at ambient conditions. At t=24 h, 300 μL of each mixture was drawn and filtered through 0.2 m, Nylon membrane by centrifugation (14000 rpm, 5 min). Each filtrate was diluted 10-fold with diluent (10/90 (v/v) ACN/MeOH) for UPLC assay. The vehicle compositions and results are shown in FIG. 2 and Table 2 below:

TABLE 2
Vehicle Compositions.

| | | TRM-201 solubility |
|---|---|---|
| Vehicle # | Vehicle Composition | (μg/mL) @ t = 24 h |
| V204 | 5/15/80 (v/w/v) DMSO / HPbCD / Water | 75.5 |
| V205 | 5/15/80 (v/w/v) EtOH / HPbCD / Water | 79.9 |
| V206 | 5/15/80 (w/w/v) Kolliphor HS 15 / HPbCD / Water | 83.1 |
| V207 | 5/15/80 (w/w/v) Poloxamer 188 / HPbCD / Water | 92.8 |
| V208 | 5/15/80 (v/w/v) PS 20 / HPbCO / Water | 88.5 |
| V209 | 5/15/80 (v/w/v) PS 80 / HPbCO / Water | 100 |
| V210 | 5/15/80 (w/w/v) Soluplus / HPbCD / Water | 111.2 |
| V211 | 5/5/15/75 (w/w/w/v) Soybean oil/ Span 40 / HPbCD / Water | 115.8 |
| V212 | 5/15/80 (v/w/v) DMSO / Captisol / Water | 95.2 |
| V213 | 5/15/80 (v/w/v) EtOH / Captisol / Water | 78.9 |
| V214 | 5/15/80 (w/w/v) Kolliphor HS 15 / Captisol / Water | 113.7 |
| V215 | 5/15/80 (w/w/v) Poloxamer 188 / Captisol / Water | 99.1 |
| V216 | 5/15/80 (v/w/v) PS 20/ Captisol / Water | 85.1 |
| V217 | 5/15/80 (v/w/v) PS 80 / Captisol / Water | 137 |
| V218 | 5/15/80 (w/w/v) Soluplus / Captisol / Water | 140.5 |
| V219 | 5/5/15/75 (w/w/w/v) Soybean oil/ Span 40 / Captisol / Water | 135.3 |

This study demonstrates that, in certain circumstances, addition of a second solubilizing agent can increase the solubility of rofecoxib in an water-based solution. Surprisingly, the addition of a second solubilizing agent differentially affected the solubility of rofecoxib in hydroxypropyl-β-cyclodextrin and sulfobutyl ether-β-cyclodextrin. For example, a 5/15/80 (w/w/v) mixture of Soluplus®/Captisol/Water (V218) achieved a solubility of rofecoxib of 140.5 μg/ml at t=24 h, whereas a 20/80 (w/v) mixture of Captisol/Water (V174) achieved a solubility of rofecoxib of 113 μg/ml at t=24 h. In some embodiments, addition of a second solubilizing agent to the pharmaceutical composition increases the solubility of a water insoluble COX-2 inhibitor by 5%, 10%, 15%, 20%, 25%, 30% or more. In contrast, a 5/15/80 (w/w/v) mixture of Soluplus®/hydroxypropyl-β-cyclodextrin/Water (V204) achieved a solubility of rofecoxib of 111.2 μg/ml at t=24 h, whereas a 20/80 (w/v) mixture of hydroxypropyl-β-cyclodextrin/Water (V138) achieved a solubility of rofecoxib of 123 μg/ml at t=24 h.

### Example 3

In some embodiments, the pharmaceutical composition described herein includes about 10 μg/ml of rofecoxib. In some embodiments, the pharmaceutical composition further includes at least one β-cyclodextrin and water. In some embodiments, the pharmaceutical composition is 10 μg/ml of rofecoxib in β-cyclodextrin and water.

In some embodiments, the pharmaceutical composition is in a 100 ml “ready to use” form. In some embodiments, the pharmaceutical composition is concentrated and has to be diluted to a 100 ml volume prior to use to create the infusate. In some embodiments, the 100 ml infusate is intravenously administered to a subject 3-4 times per day to treat acute pain in a surgical setting, for example to treat post-operative pain.

### Example 4

A study was performed to assess the solubility of rofecoxib in a number of different compositions comprising a solubilizing agent, a co-solvent helper, and/or other excipients. In some embodiments, the compositions (e.g. oral solution or composition for intravenous administration) described herein may comprise any of the following compositions or components as described in FIGS. 1 and 3 and Table 3 below.

TABLE 3
Vehicle Compositions.

| | | TRM-201 solubility |
|---|---|---|
| Vehicle # | Vehicle Composition | (μg/mL) @ t = 24 h |
| V01 | 2/98 (v/v) BnOH / Water | 9 |
| V02 | 10/90 (v/v) Corn oil / Water | 18 |
| V03 | 40/60 (v/v) Kolliphor EL/ | 294 |
| | Water | |
| V04 | 50/50 (v/v) Kolliphor EL / | 2055 |
| | EtOH | |
| V05 | 10/90 (v/v) DMSO / Water | 8 |
| V06 | 30/70 (v/v) DMA / Water | 63 |
| V07 | 1/99 (v/v) Glyceryl | 8 |
| | monooleate / Water | |
| V08 | 20% (w/v)HPbCD in water | 110 |
| V09 | 1% (w/v) Lecithin in water | 10 |
| V10 | 20/80 (v/v) NMP / Water | 40 |
| V11 | 20/80 (v/v) Miglyol 812 N / | 11 |
| | Water | |
| V12 | 1% (w/v) Oleic acid in water | 33 |
| V13 | 10% (w/v) Poloxamer 188 in | 10 |
| | water | |
| V14 | 40/60 (v/v) Water / PEG 400 | 461 |
| V15 | 10/90 (v/v) PS 20 / Water | 68 |
| V16 | 40/60 (v/v) PS 80 / Water | 367 |
| V17 | 10% (w/v) Povidone K30 in | 9 |
| | water | |
| V18 | 30/70 (v/v) Propylene glycol / | 16 |
| | Water | |
| V19 | 10/90 (v/v) Sorbitol / Water | 6 |
| V20 | 10% (w/v) Vitamin E TPGS | 157 |
| | in water | |
| V21 | 100% Water | 6 |
| V22 | 100% Ethanol | 317 |
| V23 | 50/50 (v/v) PS 80 / EtOH | 2287 |
| V24 | 50/50 (v/v) PEG 400 / EtOH | 3996 |
| V25 | 40% (w/v) Vitamin E TPGS | 2109 |
| | in EtOH | |
| V26 | 20/40/40 (v/v/v) EtOH / | 5804 |
| | Kolliphor EL / PEG 400 | |
| V27 | 20/40/40 (v/v/v) NMP / | 19408 |
| | Kolliphor EL / PEG 400 | |
| V28 | 20/40/40 (v/v/v) EtOH / | 3881 |
| | Kolliphor EL / PS 80 | |
| V29 | 20/40/40 (v/v/v) EtOH / PS | 6171 |
| | 80 / PEG 400 | |
| V30 | 20/40/40 (v/w/v) EtOH / | 6973 |
| | Vitamin E TPGS / PEG 400 | |
| V31 | 10/40/50 (v/v/v) BnOH / | 4775 |
| | EtOH / PEG 400 | |
| V32 | 10/40/50 (v/v/w) BnOH / | 545 |
| | EtOH / Decanoic acid | |
| V33 | 40% (w/v) Kolliphor HS 15 | 1819 |
| | in EtOH | |
| V34 | 20/40/40 (v/w/v) EtOH / | 6295 |
| | Kolliphor HS 15 / PEG 400 | |
| V35 | 10% (w/v) Poloxamer 188 in | 817 |
| | EtOH | |
| V36 | 100% Soybean oil | 293 |
| V37 | 10% (w/v) Span 40, 10% | 284 |
| | (w/v) Cholesterol in water | |
| V38 | 40% (w/v) Soluplus in EtOH | 1533 |
| V39 | 50/50 (v/v) Glycerin / EtOH | 489 |
| V40 | 1% (w/v) Albumin in PBS | 3 |
| | (1X) | |
| V41 | 50/50 (v/v) Transcutol / | 2215 |
| | EtOH | |
| V42 | 40/60 (v/v) EtOH / Isopropyl | 258 |
| | myristate | |
| | 10/18/36/36 (v/v/v/v) Water / | 8357 |
| | NMP / Kolliphor EL / PEG 400 | |
| V43 | 10/10/80 (v/v/v) PS 80 / PEG | 112 |
| | 400 / Water | |
| V44 | 10/10/80 (v/v/v) PS 80 / PEG | 115 |
| | 300 / Water | |
| V45 | 10/10/80 (v/v/v) Kolliphor | 96 |
| | EL / PEG 400 / Water | |
| V46 | 10/10/10/70 (v/v/v/v) EtOH / | 124 |
| | PS 80 / PEG 400 / Water | |
| V47 | 10/10/10/70 (v/v/v/v) NMP / | 160 |
| | PS 80 / PEG 400 / Water | |
| V48 | 10/10/10/70 (v/v/v/v) DMA / | 150 |
| | PS 80 / PEG 400 / Water | |
| V49 | 10/10/10/70 (v/v/v/v) | 122 |
| | Glycerin / PS 80 / PEG 400 / | |
| | Water | |
| V50 | 10/10/10/70 (v/v/v/v) PG/PS | 124 |
| | 80 / PEG 400 / Water | |
| V51 | 20/20/60 (v/v/v) PS 80 / PEG | 246 |
| | 400 / Water | |
| V52 | 20/20/60 (v/v/v) PS 80 / PEG | 228 |
| | 300 / Water | |
| V53 | V53, 10/20/20/50 (v/v/v/v) | 319 |
| | EtOH / PS 80 / PEG 400 / | |
| | Water | |
| V54 | 10/20/20/50 (v/v/v/v) NMP / | 409 |
| | PS 80 / PEG 400 / Water | |
| V55 | 10/20/20/50 (v/v/v/v) DMA / | 387 |
| | PS 80 / PEG 400 / Water | |
| V56 | 10/20/20/50 (v/v/v/v) | 288 |
| | Glycerin / PS 80 / PEG 400 / | |
| | Water | |
| V57 | 10/20/20/50 (v/v/v/v) PG / PS | 301 |
| | 80 / PEG 400 / Water | |
| V58 | 40/10/50 (v/v/v) PS 80 / PEG | 499 |
| | 400 / Water | |
| V59 | 25/25/50 (v/v/v) PS 80 / PEG | 390 |
| | 400 / Water | |
| V60 | 10/40/50 (v/v/v) PS 80 / PEG | 276 |
| | 400 / Water | |
| V61 | 60/20/20 (v/v/v) PS 80 / PEG | 2220 |
| | 400 / Water | |
| V62 | 20/60/20 (v/v/v) PS 80 / PEG | 2856 |
| | 400 / Water | |
| V63 | 10/60/30 (v/v/v) PS 80 / PEG | 1157 |
| | 400 / Water | |
| V64 | 5/1/64/30 (v/v/v/v) EtOH / PS | 1137 |
| | 80 / PEG 400 / Water | |
| V65 | 5/65/30 (v/v/v) PS 80 / PEG | 1177 |
| | 400 / Water | |
| V66 | 1/69/30 (v/v/v) PS 80 / PEG | 1180 |
| | 400 / Water | |
| V67 | 70/30 (v/v) PEG 400 / Water | 1151 |
| V68 | 10/50/40 (v/v/v) PS 80 / PEG | 501 |
| | 400 / Water | |
| V69 | 5/1/54/40 (v/v/v/v) EtOH / PS | 451 |
| | 80 / PEG 400 / Water | |
| V70 | 5/55/40 (v/v/v) PS 80 / PEG | 461 |
| | 400 / Water | |
| V71 | 1/59/40 (v/v/v) PS 80 / PEG | 438 |
| | 400 / Water | |
| V72 (V14) | 60/40 (v/v) PEG 400 / Water | 412 |
| V73 (V60) | 10/40/50 (v/v/v) PS 80 / PEG | 247 |
| | 400 / Water | |
| V74 | 5/1/44/50 (v/v/v/v) EtOH / PS | 188 |
| | 80 / PEG 400 / Water | |
| V75 | 5/45/50 (v/v/v) PS 80 / PEG | 211 |
| | 400 / Water | |
| V76 | 5/45/50 (v/v/v) EtOH / PEG | 181 |
| | 400 / Water | |
| V77 | 1/49/50 (v/v/v) PS 80 / PEG | 181 |
| | 400 / Water | |
| V78 | 50/50 (v/v) PEG 400 / Water | 170 |
| V79 | 5/1/34/60 (v/v/v/v) EtOH / PS | 100 |
| | 80 / PEG 400 / Water | |
| V80 | 5/35/60 (v/v/v) EtOH / PEG | 92 |
| | 400 / Water | |
| V81 | 1/39/60 (v/v/v) PS 80 / PEG | 97 |
| | 400 / Water | |
| V82 | 40/60 (v/v) PEG 400 / Water | 89 |
| V83 | 55/45 (v/v) PEG 400 / Water | 425 |
| V84 | 62.5/37.5 (v/v) PEG 400 / | 228 |
| | Water | |
| V85 | 100% PEG 400 | 9668 |
| V86 | 50/40/10 (v/v/v) PEG 400 / | 214 |
| | Water / Glycerin | |
| V87 | 30/70 (v/v) PEG 300 / Water | 23 |
| V88 | 5/30/65 (w/v/v) Captisol / | 47 |
| | PEG 300 / Water | |
| V89 | 5/30/65 (v/v/v) DMA / PEG | 40 |
| | 300 / Water | |
| V90 | 5/30/65 (v/v/v) DMSO / PEG | 35 |
| | 300 / Water | |
| V91 | 5/30/65 (v/v/v) EtOH / PEG | 35 |
| | 300 / Water | |
| V92 | 5/30/65 (w/v/v) HPbCD / | 49 |
| | PEG 300 / Water | |
| V93 | 5/30/65 (v/v/v) Kolliphor EL | 65 |
| | / PEG 300 / Water | |
| V94 | 5/30/65 (w/v/v) Kolliphor HS | 57 |
| | 15 / PEG 300 / Water | |
| V95 | 5/30/65 (v/v/v) NMP / PEG | 38 |
| | 300 / Water | |
| V96 | 5/30/65 (v/v/v) PG / PEG 300 | 33 |
| | / Water | |
| V97 | 5/30/65 (v/v/v) PS 20 / PEG | 67 |
| | 300 / Water | |
| V98 | 5/30/65 (v/v/v) PS 80 / PEG | 78 |
| | 300 / Water | |
| V99 | 30/70 (v/v) PEG 400 / Water | 25 |
| V100 | 5/30/65 (w/v/v) Captisol / | 48 |
| | PEG 400 / Water | |
| V101 | 5/30/65 (v/v/v) DMA / PEG | 41 |
| | 400 / Water | |
| V102 | 5/30/65 (v/v/v) DMSO / PEG | 34 |
| | 400 / Water | |
| V103 | 5/30/65 (v/v/v) EtOH / PEG | 37 |
| | 400 / Water | |
| V104 | 5/30/65 (w/v/v) HPbCD / | 55 |
| | PEG 400 / Water | |
| V105 | 5/30/65 (v/v/v) Kolliphor EL | 72 |
| | / PEG 400 / Water | |
| V106 | 5/30/65 (w/v/v) Kolliphor HS | 68 |
| | 15 / PEG 400 / Water | |
| V107 | 5/30/65 (v/v/v) NMP / PEG | 46 |
| | 400 / Water | |
| V108 | 5/30/65 (v/v/v) PG / PEG 400 | 33 |
| | / Water | |
| V109 | 5/30/65 (v/v/v) PS 20 / PEG | 65 |
| | 400 / Water | |
| V110 | 5/30/65 (v/v/v) PS 80 / PEG | 81 |
| | 400 / Water | |
| V111 | 20/80 (v/v) PEG 300 / Water | 13 |
| V112 | 5/20/75 (w/v/v) Captisol / | 33 |
| | PEG 300 / Water | |
| V113 | 5/20/75 (v/v/v) DMA / PEG | 23 |
| | 300 / Water | |
| V114 | 5/20/75 (v/v/v) DMSO / PEG | 18 |
| | 300 / Water | |
| V115 | 5/20/75 (v/v/v) EtOH / PEG | 18 |
| | 300 / Water | |
| V116 | 5/20/75 (w/v/v) HPbCD / | 39 |
| | PEG 300 / Water | |
| V117 | 5/20/75 (v/v/v) Kolliphor EL | 48 |
| | / PEG 300 / Water | |
| V118 | 5/20/75 (w/v/v) Kolliphor HS | 42 |
| | 15 / PEG 300 / Water | |
| V119 | 5/20/75 (v/v/v) NMP / PEG | 23 |
| | 300 / Water | |
| V120 | 5/20/75 (v/v/v) PG / PEG 300 | 17 |
| | / Water | |
| V121 | 5/20/75 (v/v/v) PS 20 / PEG | 53 |
| | 300 / Water | |
| V122 | 5/20/75 (v/v/v) PS 80 / PEG | 62 |
| | 300 / Water | |
| V123 | 20/80 (v/v) PEG 400 / Water | 12 |
| V124 | 5/20/75 (w/v/v) Captisol / | 39 |
| | PEG 400 / Water | |
| V125 | 5/20/75 (v/v/v) DMA / PEG | 21 |
| | 400 / Water | |
| V126 | 5/20/75 (v/v/v) DMSO / PEG | 19 |
| | 400 / Water | |
| V127 | 5/20/75 (v/v/v) EtOH / PEG | 19 |
| | 400 / Water | |
| V128 | 5/20/75 (w/v/v) HPbCD / | 43 |
| | PEG 400 / Water | |
| V129 | 5/20/75 (v/v/v) Kolliphor EL | 53 |
| | / PEG 400 / Water | |
| V130 | 5/20/75 (w/v/v) Kolliphor HS | 44 |
| | 15 / PEG 400 / Water | |
| V131 | 5/20/75 (v/v/v) NMP / PEG | 25 |
| | 400 / Water | |
| V132 | 5/20/75 (v/v/v) PG / PEG 400 | 17 |
| | / Water | |
| V133 | 5/20/75 (v/v/v) PS 20 / PEG | 48 |
| | 400 / Water | |
| V134 | 5/20/75 (v/v/v) PS 80 / PEG | 63 |
| | 400 / Water | |
| V135 | 10/90 (w/v) HPbCD / Water | 57 |
| V136 | 10/10/80 (w/v/v) HPbCD / | 56 |
| | PEG 400 / Water | |
| V137 | 10/20/70 (w/v/v) HPbCD / | 62 |
| | PEG 400 / Water | |
| V139 | 20/10/70 (w/v/v) HPbCD / | 119 |
| | PEG 400 / Water | |
| V140 | 20/20/60 (w/v/v) HPbCD / | 119 |
| | PEG 400 / Water | |
| V141(V104) | 5/30/65 (w/v/v) HPbCD / | 50 |
| | PEG 400 / Water | |
| V142 | 10/30/60 (w/v/v) HPbCD / | 79 |
| | PEG 400 / Water | |
| V143 | 15/30/55 (w/v/v) HPbCD / | 108 |
| | PEG 400 / Water | |
| V144 | 20/30/50 (w/v/v) HPbCD / | 139 |
| | PEG 400 / Water | |
| V145 | 2/5/30/63 (v/w/v/v) EtOH / | 52 |
| | HPbCD / PEG 400 / Water | |
| V146 | 2/10/30/58 (v/w/v/v) EtOH / | 84 |
| | HPbCD / PEG 400 / Water | |
| V147 | 2/15/30/53 (v/w/v/v) EtOH / | 102 |
| | HPbCD / PEG 400 / Water | |
| V148 | 2/20/30/48 (v/w/v/v) EtOH / | 142 |
| | HPbCD / PEG 400 / Water | |
| V149 | 2/5/30/63 (w/w/v/v) | 59 |
| | Kolliphor HS 15 / HPbCD / | |
| | PEG 400 / Water | |
| V150 | 2/10/30/58 (w/w/v/v) | 87 |
| | Kolliphor HS 15 / HPbCD / | |
| | PEG 400 / Water | |
| V151 | 2/15/30/53 (w/w/v/v) | 117 |
| | Kolliphor HS 15 / HPbCD / | |
| | PEG 400 / Water | |
| V152 | 2/20/30/48 (w/w/v/v) | 150 |
| | Kolliphor HS 15 / HPbCD / | |
| | PEG 400 / Water | |
| V153 | 2/5/30/63 (v/w/v/v) PS 80/ | 65 |
| | HPbCD / PEG 400 / Water | |
| V154 | 2/10/30/58 (v/w/v/v) PS 80 / | 95 |
| | HPbCD / PEG 400 / Water | |
| V155 | 2/15/30/53 (v/w/v/v) PS 80/ | 128 |
| | HPbCD / PEG 400 / Water | |
| V156 | 2/20/30/48 (v/w/v/v) PS 80 / | 147 |
| | HPbCD / PEG 400 / Water | |
| V157 | 5/5/30/60 (v/w/v/v) EtOH / | 65 |
| | HPbCD / PEG 400 / Water | |
| V158 | 5/10/30/55 (v/w/v/v) EtOH / | 89 |
| | HPbCD / PEG 400 / Water | |
| V159 | 5/15/30/50 (v/w/v/v) EtOH / | 122 |
| | HPbCD / PEG 400 / Water | |
| V160 | 5/20/30/45 (v/w/v/v) EtOH / | 146 |
| | HPbCD / PEG 400 / Water | |
| V161 | 5/5/30/60 (w/w/v/v) | 79 |
| | Kolliphor HS 15 / HPbCD / | |
| | PEG 400 / Water | |
| V162 | 5/10/30/55 (w/w/v/v) | 117 |
| | Kolliphor HS 15 / HPbCD / | |
| | PEG 400 / Water | |
| V163 | 5/15/30/50 (w/w/v/v) | 133 |
| | Kolliphor HS 15 / HPbCD / | |
| | PEG 400 / Water | |
| V164 | 5/20/30/45 (w/w/v/v) | 169 |
| | Kolliphor HS 15 / HPbCD / | |
| | PEG 400 / Water | |
| V165 | 5/5/30/60 (v/w/v/v) PS 80 / | 98 |
| | HPbCD / PEG 400 / Water | |
| V166 | 5/10/30/55 (v/w/v/v) PS 80 / | 121 |
| | HPbCD / PEG 400 / Water | |
| V167 | 5/15/30/50 (v/w/v/v) PS 80 / | 157 |
| | HPbCD / PEG 400 / Water | |
| V168 | 5/20/30/45 (v/w/v/v) PS 80 / | 202 |
| | HPbCD / PEG 400 / Water | |
| V173 | 10/90 (w/v) Captisol / Water | 55 |
| V174.5 | 10/10/80 (w/v/v) Captisol / | 56 |
| | PEG 400 / Water | |
| V180 | 10/20/70 (w/v/v) Captisol / | 59 |
| | PEG 400 / Water | |
| V181 | 20/10/70 (w/v/v) Captisol / | 102 |
| | PEG 400 / Water | |
| V182 | 20/20/60 (w/v/v) Captisol / | 101 |
| | PEG 400 / Water | |
| V183 | 10/30/60 (w/v/v) Captisol / | 68 |
| | PEG 400 / Water | |
| V184 | 15/30/55 (w/v/v) Captisol / | 85 |
| | PEG 400 / Water | |
| V185 | 20/30/50 (w/v/v) Captisol / | 104 |
| | PEG 400 / Water | |
| V186 | 2/10/30/58 (v/w/v/v) EtOH / | 74 |
| | Captisol / PEG 400 / Water | |
| V187 | 2/15/30/53 (v/w/v/v) EtOH / | 94 |
| | Captisol / PEG 400 / Water | |
| V188 | 2/20/30/48 (v/w/v/v) EtOH / | 113 |
| | Captisol / PEG 400 / Water | |
| V189 | 2/10/30/58 (w/w/v/v) | 77 |
| | Kolliphor HS 15 / Captisol / | |
| | PEG 400 / Water | |
| V190 | 2/15/30/53 (w/w/v/v) | 96 |
| | Kolliphor HS 15 / Captisol / | |
| | PEG 400 / Water | |
| V191 | 2/20/30/48 (w/w/v/v) | 123 |
| | Kolliphor HS 15 / Captisol / | |
| | PEG 400 / Water | |
| V192 | 2/10/30/58 (v/w/v/v) PS 80 / | 86 |
| | Captisol / PEG 400 / Water | |
| V193 | 2/15/30/53 (v/w/v/v) PS 80/ | 108 |
| | Captisol / PEG 400 / Water | |
| V194 | 2/20/30/48 (v/w/v/v) PS 80 / | 133 |
| | Captisol / PEG 400 / Water | |
| V195 | 5/10/30/55 (v/w/v/v) EtOH / | 85 |
| | Captisol / PEG 400 / Water | |
| V196 | 5/15/30/50 (v/w/v/v) EtOH / | 102 |
| | Captisol / PEG 400 / Water | |
| V197 | 5/20/30/45 (v/w/v/v) EtOH / | 118 |
| | Captisol / PEG 400 / Water | |
| V198 | 5/10/30/55 (w/w/v/v) | 116 |
| | Kolliphor HS 15 / Captisol / | |
| | PEG 400 / Water | |
| V199 | 5/15/30/50 (w/w/v/v) | 133 |
| | Kolliphor HS 15 / Captisol / | |
| | PEG 400 / Water | |
| V200 | 5/20/30/45 (w/w/v/v) | 149 |
| | Kolliphor HS 15 / Captisol / | |
| | PEG 400 / Water | |
| V201 | 5/10/30/55 (v/w/v/v) PS 80 / | 116 |
| | Captisol / PEG 400 / Water | |
| V202 | 5/15/30/50 (v/w/v/v) PS 80 / | 138 |
| | Captisol / PEG 400 / Water | |
| V203 | 5/20/30/45 (v/w/v/v) PS 80 / | 173 |
| | Captisol / PEG 400 / Water | |

### Example 5—Oral Solution Formulations of Rofecoxib

In some embodiments, the subject matter disclosed herein relates to oral solution formulations of water insoluble COX-2 inhibitors. In some embodiments, the water insoluble COX-2 inhibitor is selected from the group consisting of rofecoxib, etoricoxib, and celecoxib. In some embodiments, the water insoluble COX-2 inhibitor is rofecoxib. In some embodiments, the subject matter disclosed herein relates to oral solution formulations of rofecoxib.

In some embodiments, the subject matter described herein relates to a single-dose, open-label, phase I, two-period crossover pharmacokinetic study of 17.5-mg doses of TRM-201 (rofecoxib) oral tablets and a solution of TRM-201 administered as an oral solution (TRM-201 OS) to healthy subjects in a fasting state.

Study Objectives and Endpoint

Primary Objectives:

1. To evaluate and compare key pharmacokinetic parameters of the TRM 201 oral tablets and TRM 201 oral solution (TRM-201 OS). Key pharmacokinetic parameters include area under the plasma concentration-time curve from time zero to infinity (AUC0-inf) and observed maximum plasma concentration (Cₘₐₓ).

2. To assess additional pharmacokinetic parameters of TRM-201 oral solution (OS) including time to observed maximum concentration (Tₘₐₓ) and apparent terminal elimination half-life (t_{1/2}).

Secondary Objective:

3. To assess the safety and tolerability of TRM-201 OS and TRM-201 oral tablets administered to healthy subjects.

Study Design:

In some embodiments, the subject matter disclosed herein relates to a single-center, open-label, Phase I, two-period crossover pharmacokinetic (PK) study to assess single oral tablet and single oral solution administrations of TRM-201 in healthy subjects in a fasting state. Twelve heathy subjects who meet eligibility criteria will be randomized to receive one of two treatment sequences that consist of a single oral tablet of TRM-201 and a single oral solution of TRM-201 with the primary objectives of comparing the key PK parameters of the two dosage forms. Subjects will enter the clinic the day before their planned dosing and will remain in the clinic throughout the two-period crossover.

The maximum duration for each subject's participation in the study is up 35 days: up to 27 days for screening (minus admission day) and 8 in-clinic days. The 8 in-clinic days consist of 1 day for check-in and 3 days for each of the two periods plus an additional washout day between Periods 1 and 2.

Study Drug, Dosage and Route of Administration

TRM-201: An immediate-release tablet that contains rofecoxib. An oral dose of 17.5 mg rofecoxib will be administered in the two-period crossover.

TRM-201 OS (0.1 mg/mL Rofecoxib Oral Solution): An oral solution consisting of 0.1 mg/mL rofecoxib in 20% Captisol® in 50 mM citrate buffer pH 4.5 at a total dose administration of 17.5 mg of rofecoxib in 175 mL will be administered in the two-period crossover. The total amount of Captisol® to be consumed in the 175 mL oral solution is 35 g.

Patient population: healthy subjects

Number of subjects: 12

Number of Centers: 1

Test Product and Doses: TRM-201 Oral tablet, 17.5 mg and TRM-201 OS in 20% Captisol®, 17.5 mg in 175 mL

Route of administration: oral

Criteria for Inclusion/Exclusion:

Inclusions: Subjects may be included in the study only if all the following criteria are met:

1. The subject is 18 to 60 years of age, inclusive, at Screening.

2. All female subjects must have a negative pregnancy test at Screening and Check-in. Female subjects of childbearing potential must be using an acceptable method of birth control during the study (i.e., diaphragm with spermicide, intrauterine device, condom with foam or vaginal spermicide, oral contraceptives, or abstinence). Women who are surgically sterile (i.e., hysterectomy, bilateral tubal ligation, bilateral salpingectomy or bilateral oophorectomy), or postmenopausal (defined as amenorrhea for 12 consecutive months and documented serum follicle-stimulating hormone level >40 IU/mL [confirmed at Screening]) are exempt from the contraception requirement.

3. The subject has a body mass index (BMI) at Screening of 18.0 to 32.0 kg/m2, inclusive, with a minimum weight of 47 kg for women and 66 kg for men.

4. The subject is not a smoker (or user of e-cigarettes).

5. The investigator considers the subject to be in good general health as determined by medical history, clinical laboratory test results, vital sign measurements, 12-lead electrocardiogram (ECG) results, and physical examination findings at Screening and Check-in.

6. The subject agrees to comply with all protocol requirements as well as the particular requirements and specific Phase-I unit policies.

7. The subject provides written informed consent.

8. The subject has a creatinine level at or below the upper limit of normal.

Exclusions: Subjects are excluded from the study if any of the following criteria are met:

1. The subject has a history of a relevant drug allergy or food allergy/sensitivity (e.g., allergy to rofecoxib or excipients of TRM-201 (including Captisol®-containing products), allergy to other non-steroidal anti-inflammatory drugs (NSAIDs), or gluten intolerance that could preclude consumption of a standard clinic diet).

2. A female subject is pregnant or lactating.

3. The subject has a history of intolerance or hypersensitivity to aspirin or any other NSAID.

4. The subject has a positive test result for hepatitis B surface antigen, hepatitis C virus antibody, or human immunodeficiency virus types 1 or 2 antibodies at Screening.

5. The subject has used any prescription (excluding hormonal birth control) or over the counter medications (OTC) including NSAIDs (e.g., ibuprofen, naproxen, and aspirin) as well as herbal or nutritional supplements, within 14 days (or 5 half-lives, whichever is longer) before the vehicle or study drug dosing. Subjects may have taken acetaminophen (up to 2 g per day) in the 14 days prior to but not the day before dosing in this study. Prescription and OTC medications as well as herbal or nutritional supplements are prohibited throughout the study. COVID-19 vaccines are accepted concomitant medications, but subjects are not to receive a COVID vaccination within 72 hours prior to check-in.

6. The subject has any clinically significant abnormalities before dosing on Day 1 or has a history of disease, including: uncontrolled or poorly controlled hypertension; asthma or pulmonary disease; major cardiac ischemic symptoms, events, or interventions such as angina pectoris, myocardial infarction, acute coronary syndrome, decompensated congestive heart failure, coronary stent or bypass; history of cerebrovascular ischemic events (transient ischemic attack or stroke); major vascular ischemic symptoms such as intermittent claudication or vascular bypass or replacement surgery; significant cardiovascular (CV), gastrointestinal (GI), neurological, endocrine, or renal disease; hepatic impairment; cholecystectomy; other conditions known to interfere with the absorption, distribution, metabolism, or excretion of drugs; or clinically significant GI events.

7. The subject has a history or presence of any clinically significant abnormality in vital signs, ECG, or laboratory tests, or has any medical or psychiatric condition that, in the opinion of the investigator, may interfere with the study procedures or compromise subject safety (assessed at Screening and Check-in).

8. The subject is a cigarette smoker or has used nicotine or nicotine-containing products (e.g., snuff, nicotine patch, nicotine chewing gum, e-cigarettes) within 6 months prior to dosing in this study.

9. The subject has a history of alcohol abuse or drug addiction within the last year or consumes more than 1 unit (1 unit is equal to approximately ½ pint [200 mL] of beer, 1 small glass [100 mL] of wine, or 1 measure [25 mL] of spirits) of alcohol a day. Alcohol is not allowed within 7 days before study drug dosing.

10. The subject has a positive test result for drugs of abuse, alcohol, or cotinine (indicating active current smoking) at Screening.

11. The subject is a habitual and heavy coffee drinker (more than 4 cups a day, 28 cups a week).

12. The subject is involved in strenuous activity or contact sports within 24 hours prior to dosing in this study.

13. The subject has donated blood or blood products within 30 days prior to dosing in this study.

14. The subject has received study drug in another investigational study within 30 days (or less than 5 half-lives of the investigational agent) prior to dosing in this study.

15. The subject is not suitable for entry into the study, in the opinion of the investigator.

16. The subject is an employee or family member of the investigator or clinic staff.

17. The subject has evidence of current SARS-CoV-2 infection.

18. The subject has poor venous access that limits phlebotomy.

Continuing Eligibility at Check-In:

1. Females must have a negative serum pregnancy test.

2. All subjects must have a negative test result for drugs of abuse and alcohol.

3. Subjects must have had no significant changes in overall health status since screening including the use of medications.

4. The subject has not been involved in strenuous activity or contact sports within 24 hours prior to dosing in this study.

5. The subject has a creatinine level at or below the upper limit of normal.

Safety:

Safety and tolerability assessments will include monitoring and recording of adverse events (AEs), serious adverse events (SAEs), clinical laboratory assessments (hematology, serum chemistry, and urinalysis), vital sign measurements, 12-lead ECG assessments, and physical examination findings.

Any abnormal laboratory test results (hematology, serum chemistry, or urinalysis) or other safety assessments (e.g., ECGs, vital sign measurements), including those that worsen from baseline, felt to be clinically significant in the medical and scientific judgment of the investigator are to be recorded as AEs or SAEs from the time a subject signs the informed consent. Adverse events of special interest (AESIs) will be noted as follows:

Rofecoxib:
Hypertension
Congestive heart failure
Lower extremity edema
Upper GI perforations, ulcers, or bleeds
Thrombotic CV events

Pharmacokinetics:

Plasma samples will be analyzed for TRM-201 using a validated method. The obtained plasma concentration-time data for TRM-201 will be analyzed using appropriate non compartmental techniques to obtain estimates of the following parameters, where appropriate and possible: Tlag, Tmax, Cmax, AUC0-24, AUC0-last, AUC0-inf, AUCextrap, t½, lambda-z, CL/F, Vz/F.

Statistical Analysis:

Statistical analysis will be performed using SAS software Version 9.4 or later.

For categorical variables, frequencies and percentages will be presented. Continuous variables will be summarized using descriptive statistics (number of subjects, mean, standard deviation (SD), median, minimum, maximum). For PK-related data, geometric mean, geometric coefficient of variation (CV[%]) and geometric SD will also be provided.

Baseline demographic and background variables will be summarized overall for all subjects. The number of subjects who enroll in the study and the number and percentage of subjects who complete the study will be presented. The frequency and percentage of subjects who withdraw or discontinue from the study, and the reason for withdrawal or discontinuation, will also be summarized. Data will be listed in data listings.

Analysis of Pharmacokinetic Endpoints:

PK endpoints (both parameters and concentrations) will be summarized using descriptive statistics (number of subjects, mean, SD, median, minimum, maximum, geometric mean, geometric CV(%) and geometric SD). Data will be provided as data listings. Relative bioavailability will be estimated from the geometric mean ratio of Cmax, AUClast, and AUC0-inf (oral tablet versus oral solution) derived from a 2-period crossover model analysis of natural log transformed values. The associated 90% confidence interval will also be provided.

Analysis of Safety:

All safety data will be summarized, and the incidence of adverse events will be presented by the MedDRA system (Version 23.1 or a more recent version) organ class and preferred term, relationship to the test article, and severity.

Serious AEs and AEs leading to discontinuation of study drug will also be presented in the data listings.

Actual values and changes from Baseline for clinical laboratory test results, vital sign measurements, and 12-lead ECG results will be summarized at each time point using descriptive statistics (number of subjects, mean, SD, median, minimum, and maximum). Shift tables will be generated for clinical laboratory test results. Physical examination findings will be presented in a data listing.

Pharmacokinetic Results:

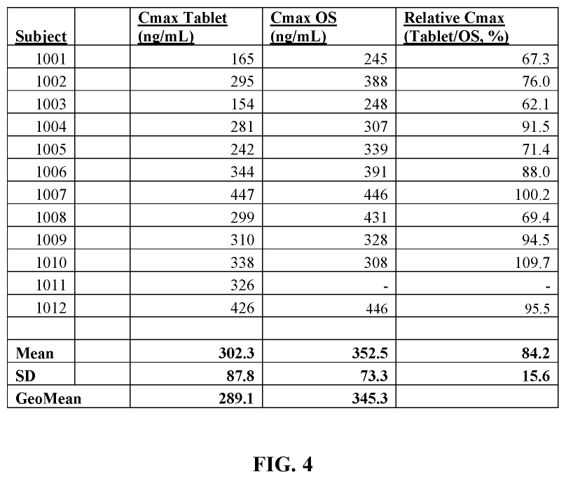

FIG. 4 shows Cₘₐₓ values for the tablet and oral solution (OS) formulations of rofecoxib (TRM-201). FIG. 4 shows that the Cₘₐₓ values for almost every subject is higher for the oral solution formulation than for the tablet formulation of rofecoxib. This leads to a higher geometric mean value of 345.3 ng/ml for Cₘₐₓ following oral solution administration compared to 289.1 ng/ml for Cₘₐₓ following administration of the tablet formulation.

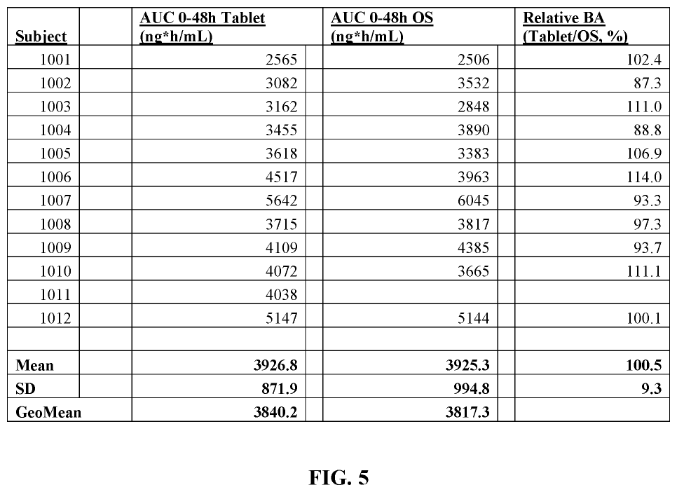

FIG. 5 shows AUC for the tablet and oral solution (OS) formulations of rofecoxib (TRM-201). FIG. 5 shows that the AUC is equivalent for the tablet formulation of rofecoxib with a geometric mean AUC at 3817.3 ng*h/ml and the oral solution formulation of rofecoxib with a geometric mean AUC at 3840.2 ng*h/ml.

What is claimed is:

1. A pharmaceutical composition comprising an aqueous solution comprising:
a) water,
b) a water insoluble COX-2 inhibitor or a pharmaceutically acceptable salt, ester or co-crystal thereof, and
c) a solubilizing agent,
wherein the solubility of the water insoluble COX-2 inhibitor in the solution is more than 10 μg/ml.

2. The pharmaceutical composition of claim 1, wherein the water insoluble COX-2 inhibitor is selected from the group consisting of rofecoxib, etoricoxib, and celecoxib.

3. The pharmaceutical composition of claim 1, wherein the water insoluble COX-2 inhibitor is rofecoxib.

4. The pharmaceutical composition of claim 1, wherein the composition further comprises at least one co-solvent helper.

5. The pharmaceutical composition of claim 1, wherein the composition further comprises at least one antioxidant.

6. The pharmaceutical composition of claim 1, wherein the composition further comprises at least one buffering agent.

7. The pharmaceutical composition of claim 1, wherein the composition further comprises at least one isotonic agent.

8. The pharmaceutical composition of claim 1, wherein said composition is a reconstituted lyophile.

9. The pharmaceutical composition of claim 8, wherein said pharmaceutical composition is diluted prior to administration.

10. The pharmaceutical composition of claim 1, wherein the solubilizing agent is a cyclodextrin.

11. The pharmaceutical composition of claim 10, wherein the cyclodextrin is selected from the group consisting of α-cyclodextrins, β-cyclodextrins, γ-cyclodextrins, and any mixtures thereof.

12. The pharmaceutical composition of claim 11, wherein the β-cyclodextrin is a hydroxypropyl-β-cyclodextrin corresponding to the CAS Registry Number 128446-35-5.

13. The pharmaceutical composition of claim 12, wherein the hydroxypropyl-β-cyclodextrin is Cavasol®.

14. The pharmaceutical composition of claim 11, wherein the β-cyclodextrin is a sulfobutyl ether-β-cyclodextrin corresponding to the CAS Registry Number 182410-00-0.

15. The pharmaceutical composition of claim 14, wherein the sulfobutyl ether-β-cyclodextrin is Captisol®.

16. The pharmaceutical composition of claim 1 or 3, having a therapeutically effective amount of the water insoluble COX-2 inhibitor in a single dose formulation, wherein the amount is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg.

17. The pharmaceutical composition of claim 1 or 3, wherein the concentration % (w/v) of the water insoluble COX-2 inhibitor in the aqueous solution is selected from the group consisting of about 0.001%, about 0.002%, about 0.003%, about 0.004%, about 0.005%, about 0.006%, about 0.007%, about 0.008%, about 0.01%, about 0.012%, about 0.015%, about 0.017%, about 0.02%, about 0.025%, about 0.03%, about 0.035%, about 0.04%, about 0.05%, about 0.06%, about 0.07% and any other suitable concentration.

18. The pharmaceutical composition of claim 1 or 3, wherein the volume of the solution is selected from the group consisting of about 5 ml, about 10 ml, about 20 ml, about 25 mL, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 180 ml, and any suitable volume.

19. The pharmaceutical composition of claim 1 or 3, wherein the solubility of the water insoluble COX-2 inhibitor in the solution is more than 20 μg/ml, more than 30 μg/ml, more than 40 μg/ml, more than 50 μg/ml, more than 60 μg/ml, more than 70 μg/ml, more than 80 μg/ml, more than 90 μg/ml, more than 100 μg/ml, more than 110 μg/ml, more than 120 μg/ml, more than 130 μg/ml, more than 140 μg/ml, or more than 150 μg/ml.

20. The pharmaceutical composition of claim 1, wherein said composition is administered as part of a combination therapy with at least one other therapeutic agent.

21. The pharmaceutical composition of claim 1, wherein said composition is suitable for intravenous administration to a subject.

22. A lyophilized pharmaceutical composition comprising a lyophilization product of any of the pharmaceutical compositions of claim 1 or 3.

23. A method for treatment of pain, fever, or inflammation in a subject in need thereof, the method comprising parenterally administering to the subject a therapeutically effective amount of a pharmaceutical composition comprising an aqueous solution comprising:
a) water,
b) a COX-2 inhibitor or a pharmaceutically acceptable salt or ester thereof, and
c) a solubilizing agent.

24. The method of claim 23, wherein the water insoluble COX-2 inhibitor is selected from the group consisting of rofecoxib, etoricoxib, and celecoxib.

25. The method of claim 24, wherein the water insoluble COX-2 inhibitor is rofecoxib.

26. The method of claim 23, wherein the composition further comprises at least one co-solvent helper.

27. The method of claim 23, wherein the composition further comprises at least one antioxidant.

28. The method of claim 23, wherein the composition further comprises at least one buffering agent.

29. The method of claim 23, wherein the composition further comprises at least one isotonic agent.

30. The method of claim 23, wherein said composition is a reconstituted lyophile.

31. The method of claim 30, wherein said pharmaceutical composition is diluted.

32. The method of claim 23, wherein the solubilizing agent is a cyclodextrin.

33. The method of claim 32, wherein the cyclodextrin is selected from the group consisting of α-cyclodextrins, β-cyclodextrins, γ-cyclodextrins, and any mixtures thereof.

34. The method of claim 33, wherein the β-cyclodextrin is a hydroxypropyl-β-cyclodextrin corresponding to the CAS Registry Number 128446-35-5.

35. The method of claim 34, wherein the hydroxypropyl-β-cyclodextrin is Cavasol®.

36. The method of claim 33, wherein the β-cyclodextrin is a sulfobutyl ether-β-cyclodextrin corresponding to the CAS Registry Number 182410-00-0.

37. The method of claim 36, wherein the sulfobutyl ether-β-cyclodextrin is Captisol®.

38. The method of claim 23 or 25, wherein the effective amount of the COX-2 inhibitor in a single dose formulation is selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg.

39. The method of claim 23 or 25, wherein the concentration % (w/v) of the COX-2 inhibitor in an aqueous formulation is selected from the group consisting of about 0.001%, about 0.002%, about 0.003%, about 0.004%, about 0.005%, about 0.006%, about 0.007%, about 0.008%, about 0.01%, about 0.012%, about 0.015%, about 0.017%, about 0.02%, about 0.025%, about 0.03%, about 0.035%, about 0.04%, about 0.05%, about 0.06%, about 0.07% and any other suitable concentration.

40. The method of claim 23 or 25, wherein the volume of an aqueous formulations of the COX-2 inhibitor is selected from the group consisting of about 1 ml, about 2 ml, about 3 ml, about 4 ml, about 5 ml, about 10 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 180 ml, and any suitable volume.

41. The method of claim 23 or 25, wherein the solubility of the COX-2 inhibitor is enhanced to more than 10 μg/ml, more than 20 μg/ml, more than 30 μg/ml, more than 40 μg/ml, more than 50 μg/ml, more than 60 μg/ml, more than 70 μg/ml, more than 80 μg/ml, more than 90 μg/ml, more than 100 μg/ml, more than 110 μg/ml, more than 120 μg/ml, more than 130 μg/ml, more than 140 μg/ml, or more than 150 μg/ml.

42. The method of claim 23, wherein said composition is administered as part of a combination therapy with at least one other therapeutic agent.

43. A lyophilized pharmaceutical composition comprising a water insoluble COX-2 inhibitor or a pharmaceutically acceptable salt, ester or co-crystal thereof and a solubilizing agent, wherein the lyophilized pharmaceutical composition is a produced by lyophilizing a pharmaceutical composition comprising an aqueous formulation comprising water, a water insoluble COX-2 inhibitor or a pharmaceutically acceptable salt, ester or co-crystal thereof, and a solubilizing agent.

44. A pharmaceutical composition comprising an oral solution comprising:
a) a COX-2 inhibitor or a pharmaceutically acceptable salt, ester or co-crystal thereof, and
b) a solubilizing agent.

45. The pharmaceutical composition of claim 44, wherein the COX-2 inhibitor is a water insoluble COX-2 inhibitor.

46. The pharmaceutical composition of claim 44, wherein the COX-2 inhibitor is selected from the group consisting of rofecoxib, etoricoxib, and celecoxib.

47. The pharmaceutical composition of claim 44, wherein the COX-2 inhibitor is rofecoxib.

48. The pharmaceutical composition of claim 44, wherein the composition further comprises at least one co-solvent helper.

49. The pharmaceutical composition of claim 44, wherein the composition further comprises at least one antioxidant.

50. The pharmaceutical composition of claim 44, wherein the composition further comprises at least one buffering agent.

51. The pharmaceutical composition of claim 44, wherein the pharmaceutical composition is diluted prior to administration.

52. The pharmaceutical composition of claim 44, wherein the solubilizing agent is a cyclodextrin.

53. The pharmaceutical composition of claim 52, wherein the cyclodextrin is selected from the group consisting of α-cyclodextrins, β-cyclodextrins, γ-cyclodextrins, and any mixtures thereof.

54. The pharmaceutical composition of claim 53, wherein the β-cyclodextrin is a hydroxypropyl-β-cyclodextrin corresponding to the CAS Registry Number 128446-35-5.

55. The pharmaceutical composition of claim 54, wherein the hydroxypropyl-β-cyclodextrin is Cavasol®.

56. The pharmaceutical composition of claim 53, wherein the β-cyclodextrin is a sulfobutyl ether-β-cyclodextrin corresponding to the CAS Registry Number 182410-00-0.

57. The pharmaceutical composition of claim 56, wherein the sulfobutyl ether-β-cyclodextrin is Captisol®.

58. The pharmaceutical composition of claim 44 or 47, wherein the pharmaceutical composition comprises an effective amount of the COX-2 inhibitor in a single dose selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg.

59. The pharmaceutical composition of claim 44 or 47, wherein the oral solution has a volume selected from the group consisting of about 5 ml, about 10 ml, about 15 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 165 ml, about 170 ml, about 175 ml, about 180 ml, about 185 ml, about 190 ml, about 200 ml, or any suitable volume.

60. The pharmaceutical composition of claim 44 or 47, wherein the oral solution comprises of about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about 0.14 mg/ml, or about 0.15 mg/ml of rofecoxib.

61. The pharmaceutical composition of claim 44, wherein the pharmaceutical composition is administered as part of a combination therapy with at least one other therapeutic agent.

62. The pharmaceutical composition of claim 44, wherein the pharmaceutical composition is suitable for oral administration to a subject.

63. The pharmaceutical composition of claim 44 or 47, wherein the oral solution comprises rofecoxib and achieves a geometric mean plasma AUC_{0-48 hr} from about 3053 to about 4772 h*ng/ml following administration of a single dose of the oral solution to a population of healthy adults less than 65 years of age.

64. The pharmaceutical composition of claim 63, wherein the geometric mean plasma AUC_{0-48 hr} is about 3053 h*ng/ml, about 3100 h*ng/ml, about 3200 h*ng/ml, about 3300 h*ng/ml, about 3400 h*ng/ml, about 3500 h*ng/ml, about 3600 h*ng/ml, about 3700 h*ng/ml, about 3800 h*ng/ml, about 3900 h*ng/ml, about 4000 h*ng/ml, about 4100 h*ng/ml, about 4200 h*ng/ml, about 4300 h*ng/ml, about 4320 h*ng/ml, about 4500 h*ng/ml, about 4600 h*ng/ml, about 4700 h*ng/ml, or about 4772 h*ng/ml.

65. The pharmaceutical composition of claim 44, wherein the oral solution comprises rofecoxib and achieves a plasma AUC_{0-48 hr} of between 174.5 h*ng/ml and 276 h*ng/ml for each 1 mg of rofecoxib in the solution.

66. The pharmaceutical composition of claim 44, wherein the oral solution comprises rofecoxib and achieves a geometric mean plasma Cₘₐₓ from about 276 to about 432 ng/ml following administration of a single dose of the oral solution to a population of healthy adults less than 65 years of age.

67. The pharmaceutical composition of claim 66, wherein the geometric mean plasma Cₘₐₓ is about 276 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, or about 432 ng/ml.

68. The pharmaceutical composition of claim 44 or 47, wherein the oral solution comprises rofecoxib and achieves a geometric mean plasma Cₘₐₓ of between 16 ng/ml and 25.1 ng/ml for each 1 mg of rofecoxib in the solution.

69. A method for treatment of pain, fever, or inflammation in a subject in need thereof, the method comprising administering to the subject a therapeutically effective amount of a pharmaceutical composition comprising an oral solution comprising:
a) a COX-2 inhibitor or a pharmaceutically acceptable salt, ester or co-crystal thereof; and
b) a solubilizing agent.

70. The pharmaceutical composition of claim 69, wherein the COX-2 inhibitor is a water insoluble COX-2 inhibitor.

71. The method of claim 69, wherein the COX-2 inhibitor is selected from the group consisting of rofecoxib, etoricoxib, and celecoxib.

72. The method of claim 69, wherein the COX-2 inhibitor is rofecoxib.

73. The method of claim 69, wherein the composition further comprises at least one co-solvent helper.

74. The method of claim 69, wherein the composition further comprises at least one antioxidant.

75. The method of claim 69, wherein the composition further comprises at least one buffering agent.

76. The method of claim 66, wherein the pharmaceutical composition is diluted prior to administration.

77. The method of claim 66, wherein the solubilizing agent is a cyclodextrin.

78. The method of claim 77, wherein the cyclodextrin is selected from the group consisting of α-cyclodextrins, β-cyclodextrins, γ-cyclodextrins, and any mixtures thereof.

79. The method of claim 78, wherein the β-cyclodextrin is a hydroxypropyl-β-cyclodextrin corresponding to the CAS Registry Number 128446-35-5.

80. The method of claim 79, wherein the hydroxypropyl-β-cyclodextrin is Cavasol®.

81. The method of claim 78, wherein the β-cyclodextrin is a sulfobutyl ether-β-cyclodextrin corresponding to the CAS Registry Number 182410-00-0.

82. The method of claim 81, wherein the sulfobutyl ether-β-cyclodextrin is Captisol®.

83. The method of claim 69 or 72, comprising an effective amount of the COX-2 inhibitor in a single dose selected from the group consisting of 2 mg, 3 mg, 5 mg, 6.25 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22.5 mg, 25 mg, 30 mg, 35 mg, 40 mg, 45 mg, 50 mg, 55 mg, 60 mg, 65 mg, and 70 mg.

84. The method of claim 69 or 72, wherein the oral solution has a volume selected from the group consisting of about 5 ml, about 10 ml, about 15 ml, about 20 ml, about 25 ml, about 30 ml, about 40 ml, about 50 ml, about 60 ml, about 70 ml, about 80 ml, about 90 ml, about 100 ml, about 110 ml, about 120 ml, about 130 ml, about 140 ml, about 150 ml, about 160 ml, about 165 ml, about 170 ml, about 175 ml, about 180 ml, about 185 ml, about 190 ml, about 200 ml, or any suitable volume.

85. The method of claim 69 or 72, wherein the oral solution comprises about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about 0.14 mg/ml, or about 0.15 mg/ml of rofecoxib.

86. The method of claim 69, wherein the composition is administered as part of a combination therapy with at least one other therapeutic agent.

87. The method of claim 69, wherein the oral solution comprises rofecoxib and achieves a geometric mean plasma AUC_{0-48 hr} from about 3053 to about 4772 h*ng/ml following administration of a single dose of the oral solution to a population of healthy adults less than 65 years of age.

88. The method of claim 87, wherein the geometric mean plasma AUC_{0-48 hr} is about 3053 h*ng/ml, about 3100 h*ng/ml, about 3200 h*ng/ml, about 3300 h*ng/ml, about 3400 h*ng/ml, about 3500 h*ng/ml, about 3600 h*ng/ml, about 3700 h*ng/ml, about 3800 h*ng/ml, about 3900 h*ng/ml, about 4000 h*ng/ml, about 4100 h*ng/ml, about 4200 h*ng/ml, about 4300 h*ng/ml, about 4320 h*ng/ml, about 4500 h*ng/ml, about 4600 h*ng/ml, about 4700 h*ng/ml, or about 4772 h*ng/ml.

89. The method of claim 69, wherein the oral solution comprises rofecoxib and achieves a plasma AUC_{0-48 hr} of between 174.5 h*ng/ml and 276 h*ng/ml for each 1 mg of rofecoxib in the solution.

90. The method of claim 69, wherein the oral solution comprises rofecoxib and achieves a geometric mean plasma Cₘₐₓ from about 276 to about 432 ng/ml following administration of a single dose of the oral solution to a population of healthy adults less than 65 years of age.

91. The method of claim 90, wherein the geometric mean plasma Cₘₐₓ is about 276 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, or about 432 ng/ml.

92. The method of claim 69, wherein the oral solution comprises rofecoxib and achieves a Cₘₐₓ of between 16 ng/ml and 25.1 ng/ml for each 1 mg of rofecoxib in the solution.
